(19) United States
(12) Patent Application Publication
(10) Pub. No.: US 2023/0165539 A1
(43) Pub. Date: Jun. 1, 2023

(54) NON-INVASIVE DETECTION OF ANOMALOUS PHYSIOLOGIC EVENTS INDICATIVE OF HYPOVOLEMIC SHOCK OF A SUBJECT

(71) Applicant: LifeLens Technologies, Inc., Ivyland, PA (US)

(72) Inventors: Landy Toth, Doylestown, PA (US); Robert S. Schwartz, Inver Grove Heights, MN (US)

(21) Appl. No.: 17/927,035

(22) PCT Filed: May 20, 2021
(86) PCT No.: PCT/US2021/033442

§ 371 Date: Nov. 22, 2022

Related U.S. Application Data
(60) Provisional application No. 63/029,098, filed on May 22, 2020.

(51) Int. Cl. A61B 5/00; A61B 5/0205

(52) U.S. Cl. CPC A61B 5/7282; A61B 5/0205; A61B 5/746; A61B 5/02438

(57) ABSTRACT

An apparatus comprises at least one processing device comprising a processor coupled to a memory. The at least one processing device is configured to obtain physiologic data from sensors associated with a wearable device of a subject, the sensors being attached to skin of the subject over vascular structures of the subject. The at least one processing device is also configured to determine differences between measured physiologic metrics in the obtained physiologic data and baseline physiologic metrics, and to detect anomalous physiologic events indicative of hypovolemic shock of the subject based at least in part on the determined differences between the measured physiologic metrics and the baseline physiologic metrics. The at least one processing device is further configured to generate notifications responsive to detecting the anomalous physiologic event indicative of hypovolemic shock of the subject, and to deliver the notifications in accordance with notification settings associated with the subject.

### TECHNICAL FIELD

The present disclosure relates to the field of physiologic monitoring and, more particularly, to devices and systems for monitoring physiologic parameters of a subject.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Hemorrhagic shock is a condition of hypovolemia resulting in poor perfusion of critical organs which is a fatal condition if not quickly resolved. Hemorrhagic shock can result from external or internal bleeding. Particularly in the case of internal bleeding, the shock may not be expected, and thus treatment delayed.

The body naturally compensates for blood loss using several mechanisms including autonomic activity, vasoconstriction, increased stroke volume, improved cardiac filling and more efficient breathing, if able. These natural compensation mechanisms can delay the onset of signs and symptoms typically diagnosed through traditionally observed metrics such as vital signs including heart rate, blood pressure, respirations, and oxygen saturation (SpO2). These traditionally used vital signs may not indicate signs of shock until a patient has lost as much as a third of their circulating blood volume.

In some environments, such as emergency rooms and obstetric delivery rooms, it can be difficult to determine when a patient has lost a critical amount of blood. For example, an emergency room receives patients from ambulance crews who may or may not accurately describe the amount of blood the patient has lost. In the case of obstetric delivery rooms, a mother's blood volume increases significantly during pregnancy and some bleeding is expected during child birth, and thus it can be difficult to discern how much blood loss is too much.

The ability to non-invasively detect early signs of hemorrhagic or hypovolemic shock would ensure that proper treatment is not delayed which would result in improved outcomes for such patients. The applications range from emergency medicine in both civilian and military settings, as well as in hospital emergency and operating rooms, intensive care units, etc. Further applications include obstetric delivery rooms where it may be difficult to differentiate between a normal or abnormal amount of blood loss during child birth.

### SUMMARY

One illustrative, non-limiting objective of this disclosure is to provide systems, devices, methods, and kits for non-invasive detection of anomalous physiologic events indicative of hemorrhagic or hypovolemic shock.

The above illustrative, non-limiting objectives are wholly or partially met by devices, systems, and methods according to the appended claims in accordance with the present disclosure. Features and aspects are set forth in the appended claims, in the following description, and in the annexed drawings in accordance with the present disclosure.

In some embodiments, an apparatus comprises at least one processing device comprising a processor coupled to a memory. The at least one processing device is configured to obtain physiologic data from one or more sensors associated with a wearable device of a subject, the one or more sensors being attached to skin of the subject over one or more vascular structures of the subject. The at least one processing device is also configured to determine differences between one or more measured physiologic metrics in the obtained physiologic data and one or more baseline physiologic metrics, and to detect one or more anomalous physiologic events indicative of hypovolemic shock of the subject based at least in part on the determined differences between the one or more measured physiologic metrics and the one or more baseline physiologic metrics. The at least one processing device is further configured to generate one or more notifications responsive to detecting the one or more anomalous physiologic event indicative of hypovolemic shock of the subject, and to deliver the one or more notifications in accordance with notification settings associated with the subject. In some embodiments, the at least one processing device is part of the wearable device. In other embodiments, the at least one processing device is coupled to the wearable device over at least one network.

The one or more sensors may be attached to the skin of the subject over one or more arteries and veins of a neck of the subject.

In some embodiments, detecting a given one of the one or more anomalous physiologic events indicative of hypovolemic shock of the subject comprises detecting pulse volume of the subject correlated with at least one of a heart rate of the subject, a pulse rate of the subject, an amplitude of a pulse of the subject, a contour of the pulse of the subject, a pulse wave of the pulse of the subject, and a timing of carotid changes relative to heart sounds of the subject.

Detecting a given one of the one or more anomalous physiologic events indicative of hypovolemic shock of the subject may also or alternatively comprise detecting at least one of a weak pulse and one or more trends in a pulse upstroke, downstroke and dwell of the subject from determined from a pulse contour of the subject.

Detecting a given one of the one or more anomalous physiologic events indicative of hypovolemic shock of the subject may further or alternatively comprise identifying one or more abnormalities in a carotid pulse of the subject. The one or more abnormalities in the carotid pulse of the subject comprise at least one of an alteration in an amplitude of a pulse peak of the carotid pulse, a distortion of a pulse upstroke of the carotid pulse, and a distortion of a pulse downstroke of the carotid pulse. Detecting the given anomalous physiologic event indicative of hypovolemic shock of the subject comprises correlating the identified one or more abnormalities in the carotid pulse of the subject with at least one of changes in respiration of the subject and changes in intrathoracic pressure of the subject.

In some embodiments, the one or more sensors attached to the skin of the subject over the one or more vascular structures of the subject are configured to independently measure: autonomic tone signals that are processed to determine heart rate variability of the subject; local electromyographic signals that are processed to determine at least one of respiration rhythm and respiration effort of the subject; oxygen saturation signals that are processed to determine a photoplethysmogram waveform characterizing changes in pulsatile arterial blood flow of the subject; movement signals that are processed to determine at least one of cardiac stroke volume and cardiac valve closures that change with variation in a pulse of the subject; at least one of electrodermal and somatosensory signals that are processed to indicate changes in autonomic activity of the subject; at least one of muscle tremor and core temperature signals that are processed to determine blood loss of the subject; and ultrasound waveform signals that are processed to determine arterial blood flow of the subject combined with changes in arterial compliance of the subject.

Detecting a given one of the one or more anomalous physiologic events indicative of hypovolemic shock of the subject may comprise identifying that a difference between at least one of the one or more measured physiologic parameters and at least one of the one or more baseline physiologic metrics exceeds at least one designated threshold specified in the notification settings associated with the subject.

Generating a given one of the one or more notifications comprises selecting a notification type of the given notification based at least in part on identifying that a difference between at least one of the one or more measured physiologic parameters and at least one of the one or more baseline physiologic metrics exceeds at least one designated threshold specified in the notification settings associated with the subject. Selecting the notification type may comprise selecting a type of stimulus to apply to the subject utilizing one or more indicator devices associated with the wearable device. The selected type of stimulus comprises at least one of an audible stimulus and a visual stimulus.

In some embodiments, delivering the one or more notifications in accordance with the notification settings associated with the subject comprises delivering the one or more notifications to at least one of the wearable device and one or more additional devices. Delivering the one or more notifications to the wearable device may comprise selecting one or more indicator devices from a set of available indicator devices associated with the wearable device, and delivering the one or more notifications via the selected one or more indicator devices. The notification settings may comprise a first threshold for delivering the one or more notifications to the wearable device and a second threshold for delivering the one or more notifications to the one or more additional devices. The one or more additional devices may comprise one or more mobile computing devices associated with one or more users responsible for monitoring a health status of the subject.

In some embodiments, a computer program product comprises a non-transitory processor-readable storage medium having stored therein executable program code which, when executed, causes at least one processing device to obtain physiologic data from one or more sensors associated with a wearable device of a subject, the one or more sensors being attached to skin of the subject over one or more vascular structures of the subject. The executable program code when executed also causes the at least one processing device to determine differences between one or more measured physiologic metrics in the obtained physiologic data and one or more baseline physiologic metrics, and to detect one or more anomalous physiologic events indicative of hypovolemic shock of the subject based at least in part on the determined differences between the one or more measured physiologic metrics and the one or more baseline physiologic metrics. The executable program code when executed further causes the at least one processing device to generate one or more notifications responsive to detecting the one or more anomalous physiologic event indicative of hypovolemic shock of the subject, and to deliver the one or more notifications in accordance with notification settings associated with the subject.

In some embodiments, a method comprises obtaining physiologic data from one or more sensors associated with a wearable device of a subject, the one or more sensors being attached to skin of the subject over one or more vascular structures of the subject. The method also comprises determining differences between one or more measured physiologic metrics in the obtained physiologic data and one or more baseline physiologic metrics, and detecting one or more anomalous physiologic events indicative of hypovolemic shock of the subject based at least in part on the determined differences between the one or more measured physiologic metrics and the one or more baseline physiologic metrics. The method further comprises generating one or more notifications responsive to detecting the one or more anomalous physiologic event indicative of hypovolemic shock of the subject, and delivering the one or more notifications in accordance with notification settings associated with the subject. The method is performed by at least one processing device comprising a processor coupled to a memory.

### BRIEF DESCRIPTION OF THE DRAWINGS

Several aspects of the disclosure can be better understood with reference to the following drawings. In the drawings, like reference numerals designate corresponding parts throughout the several views.

FIG. 1 illustrates aspects of a modular physiologic monitoring system, according to an embodiment of the invention.

FIGS. 2A-2C illustrate a modular physiologic monitoring system, according to an embodiment of the invention.

FIG. 3 illustrates a non-invasive physiologic monitoring system, according to an embodiment of the invention.

FIG. 4 illustrates a process flow for obtaining physiologic data associated with a subject utilizing a data collection module in the FIG. 3 system, according to an embodiment of the invention.

FIG. 5 illustrates information stored in a health information database in the FIG. 3 system, according to an embodiment of the invention.

FIG. 6 illustrates a process flow for processing physiologic data obtained from a subject utilizing a data processing module in the FIG. 3 system, according to an embodiment of the invention.

FIG. 7 illustrates a process flow for generating and delivering notifications utilizing a notification module in the FIG. 3 system, according to an embodiment of the invention.

FIG. 8 illustrates an image of a plot of a pulse waveform, according to an embodiment of the invention.

FIG. 9 is a flow diagram of an exemplary process for non-invasive detection of anomalous physiologic events indicative of hemorrhagic or hypovolemic shock, according to an embodiment of the invention.

### DETAILED DESCRIPTION

Particular embodiments of the present disclosure are described herein below with reference to the accompanying drawings; however, the disclosed embodiments are merely examples of the disclosure and may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure. Like reference numerals may refer to similar or identical elements throughout the description of the figures.

The accompanying drawings illustrate various embodiments of systems, methods, and embodiments of various other aspects of the disclosure. One of ordinary skill in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. It is also noted that components and elements in the figures are not necessarily drawn to scale, emphasis instead being placed upon illustrating principles.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred, systems and methods are now described.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

One illustrative, non-limiting objective of this disclosure is to provide systems, devices, methods, and kits for monitoring physiologic and/or physical signals from a subject. Another illustrative, non-limiting objective is to provide simplified systems for monitoring subjects. Another illustrative, non-limiting objective is to provide comfortable long-term wearable systems for monitoring subjects. Yet another illustrative, non-limiting objective is to provide systems for facilitating interaction between a user and a subject with regard to physiologic monitoring of the subject.

The above illustrative, non-limiting objectives are wholly or partially met by devices, systems, and methods according to the appended claims in accordance with the present disclosure. Features and aspects are set forth in the appended claims, in the following description, and in the annexed drawings in accordance with the present disclosure.

A modular physiologic monitoring system in accordance with the present disclosure is configured to monitor one or more physiologic and/or physical signals, also referred to herein as physiologic parameters, of a subject (e.g., a human subject, a patient, an athlete, a trainer, an animal such as equine, canine, porcine, bovine, etc.). The modular physiologic monitoring system may include one or more patches, each patch adapted for attachment to the body of the subject (e.g., attachable to the skin thereof, reversibly attachable, adhesively attachable, with a disposable interface and a reusable module, etc.). In aspects, the physiologic monitoring system may also include one or more modules, configured and dimensioned to mate with corresponding ones of the one or more patches, and to interface with the subject therethrough. One or more of the modules may be configured to convey and/or store one or more physiologic and/or physical signals, signals derived therefrom, and/or metrics derived therefrom obtained via the interface with the subject.

Each module may include a power source (e.g., a battery, a rechargeable battery, an energy harvesting transducer, microcircuit, an energy reservoir, a thermal gradient harvesting transducer, a kinetic energy harvesting transducer, a radio frequency energy harvesting transducer, a fuel cell, a biofuel cell, etc.), signal conditioning circuitry, communication circuitry, one or more sensors, or the like, configured to generate one or more signals (e.g., physiologic and/or physical signals), stimulus, etc.

One or more of the patches may include one or more interconnects, configured and dimensioned so as to couple with one or more of the modules, said modules including a complementary interconnect configured and dimensioned to couple with the corresponding patch. The patch may include a bioadhesive interface for attachment to the subject, the module retainable against the subject via interconnection with the patch.

In aspects, the patch may be configured so as to be single use (e.g., disposable). The patch may include a thin, breathable, stretchable laminate. In aspects, the laminate may include a substrate, a bioadhesive, one or more sensing or stimulating elements in accordance with the present disclosure, and one or more interconnects for coupling one or more of the sensing elements with a corresponding module.

In aspects, to retain a high degree of comfort and long term wearability of the patch on a subject, to limit interference with normal body function, to limit interference with joint movement, or the like, the patch may be sufficiently thin and frail, such that it may not substantially retain a predetermined shape while free standing. Such a definition is described in further detail below. The patch may be provided with a temporary stiffening film to retain the shape thereof prior to placement of the patch onto the body of a subject. Once adhered to the subject, the temporary stiffening film may be removed from the patch. While the patch is adhered to the subject, the shape and functionality of the patch may be substantially retained. Upon removal of the patch from the subject, the now freestanding patch is sufficiently frail such that the patch can no longer substantially retain the predetermined shape (e.g., sufficiently frail such that the patch will not survive in a free standing state). In aspects, stretch applied to the patch while removing the patch from the subject may result in snap back once the patch is in a freestanding state that renders such a patch to crumple into a ball and no longer function. Removal of the patch interface from the skin of the subject may result in a permanent loss in shape of the patch interface without tearing of the patch interface. In aspects, the interconnect may be sufficiently frail such that removal of the patch interface from the skin of the subject may result in a permanent loss of shape of the interconnect.

In aspects, the patch may include a film (e.g., a substrate), with sufficiently high tear strength, such that, as the patch is peeled from the skin of a subject, the patch does not tear. In aspects, the ratio between the tear strength of the patch and the peel adhesion strength of the patch to skin (e.g., tear strength: peel adhesion strength), is greater than 8:1, greater than 4:1, greater than 2:1, or the like. Such a configuration may be advantageous so as to ensure the patch may be easily and reliably removed from the subject after use without tearing.

In aspects, the patch may include a bioadhesive with peel tack to mammalian skin of greater than 0.02 Newtons per millimeter (N/mm), greater than 0.1 N/mm, greater than 0.25 N/mm, greater than 0.50 N/mm, greater than 0.75 N/mm, greater than 2 N/mm, or the like. Such peel tack may be approximately determined using an American Society for Testing and Materials (ASTM) standard test, ASTM D3330: Standard test method for peel adhesion of pressure-sensitive tape.

In aspects, the patch may exhibit a tear strength of greater than 0.5 N/mm, greater than 1 N/mm, greater than 2N/mm, greater than 8 N/mm, or the like. Such tear strength may be approximately determined using an ASTM standard test, ASTM D624: Standard test method for tear strength of conventional vulcanized rubber and thermoplastic elastomers. In aspects, a patch interface in accordance with the present disclosure may have a ratio between the tear strength of the patch and the peel tack of the adhesive to mammalian skin is greater than 8:1, greater than 4:1, greater than 2:1, or the like.

In aspects, the patch may be provided with a characteristic thickness of less than 50 micrometer (μm), less than 25 μm, less than 12 μm, less than 8 μm, less than 4 μm, or the like. Yet, in aspects, a balance between the thickness, stiffness, and tear strength may be obtained so as to maintain sufficiently high comfort levels for a subject, minimizing skin stresses during use (e.g., minimizing skin stretch related discomfort and extraneous signals as the body moves locally around the patch during use), minimizing impact on skin health, minimizing risk of rucking during use, and minimizing risk of maceration to the skin of a subject, while limiting risk of tearing of the patch during removal from a subject, etc.

In aspects, the properties of the patch may be further altered so as to balance the hydration levels of one or more hydrophilic or amphiphilic components of the patch while attached to a subject. Such adjustment may be advantageous to prevent over hydration or drying of an ionically conducting component of the patch, to manage heat transfer coefficients within one or more elements of the patch, to manage salt retention into a reservoir in accordance with the present disclosure, and/or migration during exercise, to prevent pooling of exudates, sweat, or the like into a fluid measuring sensor incorporated into the patch or associated module, etc. In aspects, the patch or a rate determining component thereof may be configured with a moisture vapor transmission rate of between 200 grams per meter squared per 24 hours (g/m²/24 hrs) and 20,000 g/m²/24 hrs, between 500 g/m²/24 hrs and 12,000 g/m²/24 hrs, between 2,000 g/m²/24 hrs and 8,000 g/m²/24 hrs, or the like.

Such a configuration may be advantageous for providing a comfortable wearable physiologic monitor for a subject, while reducing material waste and/or cost of goods, preventing contamination or disease spread through uncontrolled re-use, and the like.

In aspects, one or more patches and/or modules may be configured for electrically conducting interconnection, inductively coupled interconnection, capacitively coupled interconnection, with each other. In the case of an electrically conducting interconnect, each patch and module interconnect may include complementary electrically conducting connectors, configured and dimensioned so as to mate together upon attachment. In the case of an inductively or capacitively coupled interconnect, the patch and module may include complementary coils or electrodes configured and dimensioned so as to mate together upon attachment.

Each patch or patch-module pair may be configured as a sensing device to monitor one or more local physiologic and/or physical parameters of the attached subject (e.g., local to the site of attachment, etc.), local environment, combinations thereof, or the like, and to relay such information in the form of signals to a host device (e.g., via a wireless connection, via a body area network connection, or the like), one or more patches or modules on the subject, or the like. Each patch and/or patch-module pair may also or alternatively be configured as a stimulating device to apply a stimulus to the subject in response to signaling from the host device, the signaling being based on analysis of the physiologic and/or physical parameters of the subject measured by the sensing device(s).

In aspects, the host device may be configured to coordinate information exchange to/from each module and/or patch, and to generate one or more physiologic signals, physical signals, environmental signals, kinetic signals, diagnostic signals, alerts, reports, recommendation signals, commands, combinations thereof, or the like for the subject, a user, a network, an electronic health record (EHR), a database (e.g., as part of a data management center, an EHR, a social network, etc.), a processor, combinations thereof, or the like. In aspects, the host device may include features for recharging and/or performing diagnostic tests on one or more of the modules. In aspects, a host device in accordance with the present disclosure may be integrated into a bedside alarm clock, housed in an accessory, within a purse, a backpack, a wallet, or may be included in a mobile computing device, a smartphone, a tablet computer, a pager, a laptop, a local router, a data recorder, a network hub, a server, a secondary mobile computing device, a repeater, a combination thereof, or the like.

In aspects, a system in accordance with the present disclosure may include a plurality of substantially similar modules (e.g., generally interchangeable modules, but with unique identifiers), for coupling with a plurality of patches, each patch, optionally different from the other patches in the system (e.g., potentially including alternative sensors, sensor types, sensor configurations, electrodes, electrode configurations, etc.). Each patch may include an interconnect suitable for attachment to an associated module. Upon attachment of a module to a corresponding patch, the module may validate the type and operation of the patch to which it has been mated. In aspects, the module may then initiate monitoring operations on the subject via the attached patch, communicate with one or more other patches on the subject, a hub, etc. The data collection from each module may be coordinated through one or more modules and/or with a host device in accordance with the present disclosure. The modules may report a timestamp along with the data in order to synchronize data collection across multiple patch-module pairs on the subject, between subjects, etc. Thus, if a module is to be replaced, a hot swappable replacement (e.g., replacement during a monitoring procedure) can be carried out easily by the subject, a caregiver, practitioner, etc., during the monitoring process. Such a configuration may be advantageous for performing redundant, continuous monitoring of a subject, and/or to obtain spatially relevant information from a plurality of locations on the subject during use.

In aspects, the modules and/or patches may include corresponding interconnects for coupling with each other during use. The interconnects may include one or more connectors, configured such that the modules and patches may only couple in a single unique orientation with respect to each other. In aspects, the modules may be color coded by function. A temporary stiffening element attached to a patch may include instructions, corresponding color coding, etc., so as to assist a user or subject with simplifying the process of monitoring.

In addition to physiologic monitoring, one or more patches and/or modules may be used to provide a stimulus to the subject, as will be described in further detail below.

According to aspects, there is provided use of a modular physiologic monitoring system in accordance with the present disclosure to monitor a subject, to monitor an electrocardiogram (EKG) of a subject, to perform one or more tasks in accordance with the present disclosure, etc.

According to aspects, there is provided an interface (e.g., a patch in accordance with the present disclosure) for monitoring a physiologic, physical, and/or electrophysiological signal from a subject. The interface or patch may include a substrate, an adhesive coupled to the substrate formulated for attachment to the skin of a subject, and one or more sensors and/or electrodes each in accordance with the present disclosure coupled to the substrate, arranged, configured, and dimensioned to interface with the subject. The substrate may be formed from an elastic or polymeric material, such that the patch is configured to maintain operation when stretched to more than 25%, more than 50%, or more than 80%.

According to aspects, there is provided an isolating patch for providing a barrier between a handheld monitoring device with a plurality of contact pads and a subject, including a flexible substrate with two surfaces, a patient facing surface and an opposing surface, and an electrically and/or ionically conducting adhesive coupled to at least a portion of the patient facing surface configured so as to electrically and mechanically couple with the subject when placed thereupon, wherein the conducting adhesive is exposed within one or more regions of the opposing surface of the substrate, the regions patterned so as to substantially match the dimensions and layout of the contact pads. In aspects, the conducting adhesive may include an anisotropically conducting adhesive, with the direction of conduction oriented substantially normal to the surfaces of the substrate.

In aspects, the adhesive may be patterned onto the substrate so as to form one or more exposed regions of the substrate, one or more of the sensors and/or electrodes arranged within the exposed regions. One or more of the electrodes may include an inherently or ionically conducting gel adhesive.

In aspects, one or more of the electrodes may include an electrode feature arranged so as to improve the electrical connection between the electrode and the skin upon placement on a subject. In aspects, the improved electrical connection may be achieved after pressure is applied to the electrode (e.g., after the patch is secured to the subject and then a pressure is applied to the electrode). The electrode feature may include one or more microfibers, barbs, microneedles, or spikes to penetrate into a stratum corneum of the skin. The electrode feature may be configured to penetrate less than 2 mm into the skin, less than 1 mm, less than 0.5 mm, less than 0.2 mm, or the like during engagement therewith. In aspects, a gel adhesive in accordance with the present disclosure located adjacent to the electrode features (e.g., between the features and the skin) may be configured to maintain the improved electrical connection to the skin for more than 1 hour, more than 1 day, or more than 3 days after the electrode contacts the skin or pressure is applied to the electrode.

In aspects, a patch interface in accordance with the present disclosure may include one or more stretchable electrically conducting traces attached to the substrate, arranged so as to couple one or more of the sensors and/or electrodes with one or more of the interconnects.

In aspects, the interconnect may include a plurality of connectors, the connectors physically connected to each other through the substrate. The patch may include an isolating region arranged so as to isolate one or more of the connectors from the skin while the patch is engaged therewith.

According to aspects, there is provided a device (e.g., a module in accordance with the present disclosure) for monitoring a physiologic, physical, and/or electrophysiological signal from a subject. The module may include a housing, a printed circuit board (PCB) including one or more microcircuits, and an interconnect configured for placement of the device onto a subject interface (e.g., a patch in accordance with the present disclosure). The printed circuit board may constitute at least a portion of the housing in some embodiments. The module may include a three-dimensional antenna coupled to the microcircuits (e.g., coupled with a transceiver, transmitter, radio, etc., included within the microcircuits). In aspects, the antenna may be printed onto or embedded into the housing. In aspects, the antenna may be printed on an interior wall of or embedded into the housing, the circuit board providing a ground plane for the antenna. In aspects, the housing may be shaped like a dome and the antenna may be patterned into a spiraling helix centered within the dome.

In aspects, a module in accordance with the present disclosure may include a sensor coupled with one or more of the microcircuits, the sensor configured to interface with the subject upon attachment of the module to the patch interface. The module may include a sensor and/or microelectronics configured to interface with a sensor included on a corresponding patch interface. In aspects, one or more of the sensors may include an electrophysiologic sensor, a temperature sensor, a thermal gradient sensor, a barometer, an altimeter, an accelerometer, a gyroscope, a humidity sensor, a magnetometer, an inclinometer, an oximeter, a colorimetric monitor, a sweat analyte sensor, a galvanic skin response sensor, an interfacial pressure sensor, a flow sensor, a stretch sensor, a microphone, a combination thereof, or the like.

In aspects, the module may be hermetically sealed. The module and/or patch interface may include a gasket coupled to the circuit board or the substrate, the gasket formed so as to isolate the region formed by the module interconnect and the patch from a surrounding environment, when the module is coupled with the patch.

In aspects, the module interconnect may include an electrically conducting magnetic element, and the patch interface may include one or more ferromagnetic regions coupled to the substrate, the magnetic elements arranged so as to physically and/or electrically couple the module to the patch interface when the magnetic elements are aligned with the ferromagnetic regions. In aspects, the ferromagnetic regions may be formed from stretchable pseudo elastic material and/or may be printed onto the substrate. In aspects, the module and/or the patch interface may include one or more fiducial markings to visually assist with the alignment of the module to the patch during coupling thereof.

According to aspects, there is provided a kit for monitoring a physiologic, physical, and/or electrophysiological signal from a subject, including one or more patches in accordance with the present disclosure, one or more modules in accordance with the present disclosure, a recharging bay in accordance with the present disclosure, and one or more accessories in accordance with the present disclosure. One or more of the accessories may include an adhesive removing agent configured to facilitate substantially pain free removal of one or more of the patches from a subject.

According to aspects, there is provided a service system for managing the collection of physiologic data from a customer, including a customer data management service, configured to generate and/or store the customer profile referencing customer preferences, data sets, and/or monitoring sessions, an automated product delivery service configured to provide the customer with one or more monitoring products or supplies in accordance with the present disclosure, and a datacenter configured to store, analyze, and/or manage the data obtained from the customer during one or more monitoring sessions.

In aspects, the service system may include a report generating service configured to generate one or more monitoring reports based upon the data obtained during one or more monitoring sessions, a report generating service coupled to the datacenter configured to generate one or more monitoring reports based upon the data obtained during one or more monitoring sessions, and/or a recurrent billing system configured to bill the customer based upon the number or patches consumed, the data stored, and/or the reports generated throughout the course of one or more monitoring sessions.

According to aspects, there is provided a method for monitoring one or more physiologic and/or electrophysiological signals from a subject, including attaching one or more soft breathable and hypoallergenic devices to one or more sites on the subject, obtaining one or more local physiologic and/or electrophysiological signals from each of the devices, and analyzing the signals obtained from each of the devices to generate a metric, diagnostic, report, and/or additional signals therefrom.

In aspects, the method may include hot swapping one or more of the devices without interrupting the step of obtaining, and/or calibrating one or more of the devices while on the subject. In aspects, the step of calibrating may be performed with an additional medical device (e.g., a blood pressure cuff, a thermometer, a pulse oximeter, a cardiopulmonary assessment system, a clinical grade EKG diagnostic system, etc.).

In aspects, the method may include determining the position and/or orientation of one or more of the devices on the subject, and/or determining the position and/or orientation from a photograph, a video, or a surveillance video.

In aspects, one or more steps of a method in accordance with the present disclosure may be performed at least in part by a device, patch interface, module, and/or system each in accordance with the present disclosure.

According to aspects, there is provided a system for measuring blood pressure of a subject in an ambulatory setting including an EKG device in accordance with the present disclosure (e.g., a patch/module pair in accordance with the present disclosure configured to measure local electrophysiological signals in adjacent tissues), configured for placement onto a torso of the subject, the EKG device configured to measure an electrocardiographic signal from the torso of the subject so as to produce an EKG signal, one or more pulse devices (e.g., patch/module pairs in accordance with the present disclosure configured to measure local blood flow in adjacent tissues) each in accordance with the present disclosure, configured for placement onto one or more sites on one or more extremities of the subject, each of the pulse devices configured to measure a local pulse at the placement site so as to produce one or more pulse signals; and a processor included in or coupled to one or more of the EKG device and the pulse devices, the processor configured to receive the EKG signal, the pulse signals, and/or signals generated therefrom, the processor including an algorithm, the algorithm configured to analyze one or more temporal metrics from the signals in combination with one or more calibration parameters, to determine the blood pressure of the subject.

In aspects, the system for monitoring blood pressure of a subject may include a blood pressure cuff configured to produce a calibration signal, the processor configured to generate one or more of the calibration parameters, from the calibration signal in combination with the EKG signal, and pulse signals.

In aspects, one or more of the devices may include an orientation sensor, the orientation sensor configured to obtain an orientation signal, the processor configured to receive the orientation signal or a signal generated therefrom, and to incorporate the orientation signal into the analysis. Some non-limiting examples of orientation sensors include one or more of an altimeter, a barometer, a tilt sensor, a gyroscope, combinations thereof, or the like.

A system for measuring the effect of an impact on physiologic state of a subject including an electroencephalogram (EEG) device (e.g., a patch/module pair in accordance with the present disclosure configured to measure local electrophysiological signals associated with brain activity in adjacent tissues) in accordance with the present disclosure, configured for placement behind an ear, on the forehead, near a temple, onto the neck of the subject, or the like, the EEG device configured to measure an electroencephalographic signal from the head of the subject so as to produce an EEG signal, and configured to measure one or more kinetic and/or kinematic signals from the head of the subject so as to produce an impact signal, and a processor included in or coupled to the EEG device, the processor configured to receive the EEG signal, the impact signals, and/or signals generated therefrom, the processor including an algorithm, the algorithm configured to analyze the impact signals to determine if the subject has suffered an impact, to separate the signals into pre impact and post impact portions and to compare the pre and post impact portions of the EEG signal, to determine the effect of the impact on the subject.

In aspects, the EEG device may include additional sensors such as a temperature sensor configured to generate a temperature signal from the subject or a signal generated therefrom, the processor configured to receive the temperature signal and to assess a thermal state of the subject therefrom. In aspects, the EEG device may include a hydration sensor configured to generate a fluid level signal from the subject, the processor configured to receive the fluid level signal or a signal generated therefrom, and to assess the hydration state of the subject therefrom.

In aspects, the EEG device and/or the processor may include or be coupled to a memory element, the memory element including sufficiently large space to store the signals for a period of 3 minutes, 10 minutes, 30 minutes, or 1 hour.

In aspects, the system for measuring the effect of an impact on physiologic state of a subject may include an EKG device (e.g., a patch/module pair in accordance with the present disclosure configured to measure local electrophysiological signals in adjacent tissues) in accordance with the present disclosure, the EKG device configured for placement onto the torso or neck of the subject, the EKG device configured to measure an electrophysiological signal pertaining to cardiac function of the subject so as to produce an EKG signal, the processor configured to receive the EKG signal or a signal generated therefrom, the algorithm configured so as to incorporate the EKG signal into the assessment. In aspects, the processor may be configured to extract a heart rate variability (HRV) signal from the EKG signal, a pre impact and post impact portion of the HRV signal compared to determine at least a portion of the effect of the impact.

According to aspects, there is provided a system for assessing a sleep state of a subject including an electromyography (EMG)/electrooculography (EOG) device (e.g., a patch/module pair in accordance with the present disclosure configured to measure local electromyographic and/or electrooculographic signals from adjacent tissues), in accordance with the present disclosure, configured for placement behind an ear, on a forehead, substantially around an eye, near a temple, or onto a neck of the subject, the EMG/EOG device configured to measure one or more electromyographic and/or electrooculographic signals from the head or neck of the subject so as to produce an EMG/EOG signal, and a processor included in or coupled to the EMG/EOG device, the processor configured to receive the EMG/EOG signal, and/or signals generated therefrom, the processor including an algorithm, the algorithm configured to analyze EMG/EOG signal, to determine the sleep state of the subject.

In aspects, the EMG/EOG device may include a microphone, the microphone configured to obtain an acoustic signal from the subject, the processor configured to receive the acoustic signal or a signal generated therefrom, the algorithm configured so as to incorporate the acoustic signal into the assessment.

In aspects, the system may include a sensor for evaluating oxygen saturation (SpO2) at one or more sites on the subject to obtain an oxygen saturation signal from the subject, the processor configured to receive the oxygen saturation signal or a signal generated therefrom, the algorithm configured so as to incorporate the oxygen saturation signal into the assessment.

In aspects, the processor may include a signal analysis function, the signal analysis function configured to analyze the EMG/EOG signals, the acoustic signal, and/or the oxygen saturation signal to determine the sleep state of the subject, identify snoring, identify a sleep apnea event, identify a bruxism event, identify a rapid eye movement (REM) sleep state, identify a sleep walking state, a sleep talking state, a nightmare, or identify a waking event. In aspects, the system may include a feedback mechanism, configured to interact with the subject, a user, a doctor, a nurse, a partner, a combination thereof, or the like. The processor may be configured to provide a feedback signal to the feedback mechanism based upon the analysis of the sleep state of the subject. The feedback mechanism may include a transducer, a loudspeaker, tactile actuator, a visual feedback means, a light source, a buzzer, a combination thereof, or the like to interact with the subject, the user, the doctor, the nurse, the partner, or the like.

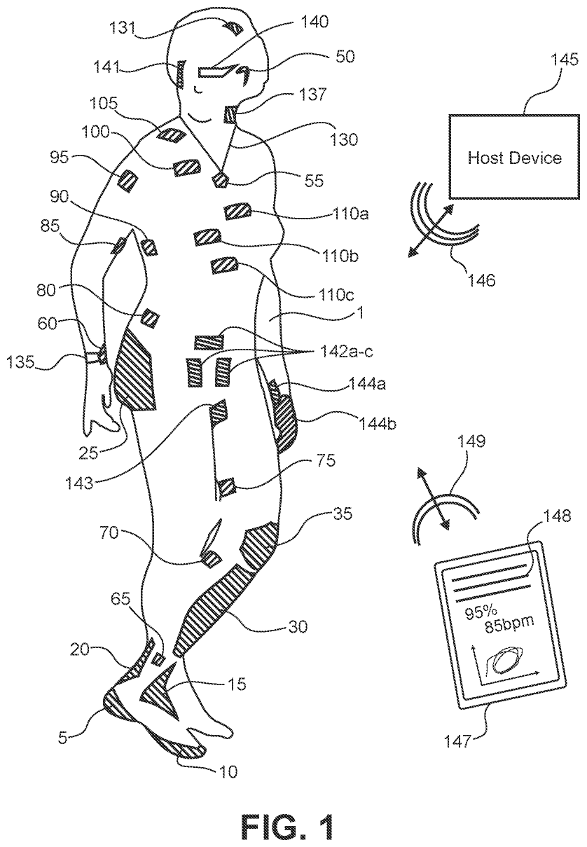

A modular physiologic monitoring system, in some embodiments, includes one or more sensing devices, which may be placed or attached to one or more sites on the subject. Alternatively or additionally, one or more sensing devices may be placed “off” the subject, such as one or more sensors (e.g., cameras, acoustic sensors, etc.) that are not physically attached to the subject. The sensing devices are utilized to establish whether or not an event is occurring and to determine one or more characteristics of the event by monitoring and measuring physiologic parameters of the subject. The determination of whether an event has occurred or is occurring may be made by a device that is at least partially external and physically distinct from the one or more sensing devices, such as a host device in wired or wireless communication with the sensing devices as described below with respect to FIG. 1. The modular physiologic monitoring system includes one or more stimulating devices, which again may be any combination of devices that are attached to the subject or placed “off” the subject, to apply a stimulus to the subject in response to a detected event. Various types of stimulus may be applied, including but not limited to stimulating via thermal input, vibration input, mechanical input, a compression or the like with an electrical input, etc.

The sensing devices of a modular physiologic monitoring system, such as patch-module pairs described below with respect to FIG. 1, may be used to monitor one or more physiologic functions or parameters of a subject, as will be described in further detail below. The sensing devices of the modular physiologic monitoring system, or a host device configured to receive data or measurements from the sensing devices, may be utilized to monitor for one or more events (e.g., through analysis of signals measured by the sensing devices, from metrics derived from the signals, etc.). The stimulating devices of the modular physiologic monitoring system may be configured to deliver one or more stimuli (e.g., electrical, vibrational, acoustic, visual, etc.) to the subject. The stimulating devices may receive a signal from one or more of the sensing devices or a host device, and provide the stimulation in response to the received signal.

FIG. 1 shows aspects of a modular physiologic monitoring system in accordance with the present disclosure. In FIG. 1, a subject 1 is shown with a number of patches and/or patch-module pairs each in accordance with the present disclosure attached thereto at sites described below, a host device 145 in accordance with the present disclosure, a feedback/user device 147 in accordance with the present disclosure displaying some data 148 based upon signals obtained from the subject 1, and one or more feedback devices 135, 140, in accordance with the present disclosure configured to convey to the subject 1 one or more aspects of the signals or information gleaned therefrom. In some embodiments, the feedback devices 135, 140 may also or alternatively function as stimulating devices. The host device 145, the user device 147, the patches and/or patch-module pairs, and/or the feedback devices 135, 140 may be configured for wireless communication 146, 149 during a monitoring session.

In aspects, a patch-module pair may be adapted for placement almost anywhere on the body of a subject 1. As shown in FIG. 1, some sites may include attachment to the cranium or forehead 131, the temple, the ear or behind the ear 50, the neck, the front, side, or back of the neck 137, a shoulder 105, a chest region with minimal muscle mass 100, integrated into a piece of ornamental jewelry 55 (may be a host, a hub, a feedback device, etc.), arrangement on the torso 110a-c, arrangement on the abdomen 80 for monitoring movement or breathing, below the rib cage 90 for monitoring respiration (generally on the right side of the body to substantially reduce EKG influences on the measurements), on a muscle such as a bicep 85, on a wrist 135 or in combination with a wearable computing device 60 on the wrist (e.g., a smart watch, a fitness band, etc.), on a buttocks 25, on a thigh 75, on a calf muscle 70, on a knee 35 particularly for proprioception based studies and impact studies, on a shin 30 primarily for impact studies, on an ankle 65, over an Achilles tendon 20, on the front or top of the foot 15, on a heel 5, or around the bottom of a foot or toes 10. Other sites for placement of such devices are envisioned. Selection of the monitoring and/or stimulating sites is generally determined based upon the intended application of the patch-module pairs described herein.

Additional placement sites on the abdomen, perineal region 142a-c, genitals, urogenital triangle, anal triangle, sacral region, inner thigh 143, or the like may be advantageous in the assessment of autonomic neural function of a subject. Such placements regions may be advantageous for assessment of parasympathetic nervous system (PNS) activity, somatosensory function, assessment of sympathetic nervous system (SNS) functionality, etc.

Placement sites on the wrist 144a, hand 144b or the like may advantageous for interacting with a subject, such as via performing a stress test, performing a thermal stress test, performing a tactile stress test, monitoring outflow, afferent traffic, efferent traffic, etc.

Placement sites on the nipples, areola, lips, labia, clitoris, penis, the anal sphincter, levator ani muscle, over the ischiocavernous muscle, deep transverse perineal muscle, labium minus, labium majus, one or more nerves near the surface thereof, posterior scrotal nerves, perineal membrane, perineal nerves, superficial transverse perineal nerves, dorsal nerves, inferior rectal nerves, etc., may be advantageous for assessment of autonomic neural ablation procedures, autonomic neural modulation procedures, assessment of the PNS of a subject, assessment of sexual dysfunction of a subject, etc.

Placement sites on the face 141, over ocular muscles, near the eye, over a facial muscle (e.g., a nasalis, temporalis, zygonaticus minor/major, orbicularis oculi, occipitofrontalis), near a nasal canal, over a facial bone (e.g., frontal process, zygomatic bone/surface, zygomaticofacial foreman, malar bone, nasal bone, frontal bone, maxilla, temporal bone, occipital bone, etc.), may be advantageous to assess ocular function, salivary function, sinus function, interaction with the lips, interaction with one or more nerves of the PNS (e.g., interacting with the vagus nerve within, on, and/or near the ear of the subject), etc.

In aspects, a system in accordance with the present disclosure may be configured to monitor one or more physiologic parameters of the subject 1 before, during, and/or after one or more of, a stress test, consumption of a medication, exercise, a rehabilitation session, a massage, driving, a movie, an amusement park ride, sleep, intercourse, a surgical, interventional, or non-invasive procedure, a neural remodeling procedure, a denervation procedure, a sympathectomy, a neural ablation, a peripheral nerve ablation, a radio-surgical procedure, an interventional procedure, a cardiac repair, administration of an analgesic, a combination thereof, or the like. In aspects, a system in accordance with the present disclosure may be configured to monitor one or more aspects of an autonomic neural response to a procedure, confirm completion of the procedure, select candidates for a procedure, follow up on a subject after having received a procedure, assess the durability of a procedure, or the like (e.g., such as wherein the procedure is a renal denervation procedure, a carotid body denervation procedure, a hepatic artery denervation procedure, a LUTs treatment, a bladder denervation procedure, a urethral treatment, a prostate ablation, a prostate nerve denervation procedure, a cancer treatment, a pain block, a neural block, a bronchial denervation procedure, a carotid sinus neuromodulation procedure, implantation of a neuromodulation device, tuning of a neuromodulation device, etc.).

Additional details regarding modular physiologic monitoring systems, kits and methods are further described in PCT application serial no. PCT/US2014/041339, published as WO 2014/197822 and titled “Modular Physiologic Monitoring Systems, Kits, and Methods,” PCT application serial no. PCT/US2015/043123, published as WO 2016/019250 and titled “Modular Physiologic Monitoring Systems, Kits, and Methods,” and PCT application serial no. PCT/US2017/030186, published as WO 2017/190049 and titled “Monitoring and Management of Physiologic Parameters of a Subject,” the disclosures of which are incorporated by reference herein in their entirety.

In some embodiments, modular physiologic monitoring systems may include sensing and stimulating devices that are physically distinct, such as sensing and stimulating devices that are physically attached to a subject at varying locations. For example, the sensing and stimulating devices may include different ones of the patch-module pairs described above with respect to FIG. 1. In other embodiments, one or more devices may provide both monitoring and stimulating functionality. For example, one or more of the patch-module pairs described above with respect to FIG. 1 may be configured to function as both a sensing device and a stimulating device. It is to be appreciated, however, that embodiments are not limited solely for use with the patch-module pairs of FIG. 1 as sensing and stimulating devices. Various other types of sensing and stimulating devices may be utilized, including but not limited to sensors that are “off-body” with respect to subject 1.

The sensing and/or stimulating devices of a modular physiologic monitoring system may be configured for radio frequency (RF) or other wireless and/or wired connection with one another and/or a host device. Such RF or other connection may be used to transmit or receive feedback parameters or other signaling between the sensing and stimulating devices. The feedback, for example, may be provided based on measurements of physiologic parameters that are obtained using the sensing devices to determine when events related to cardiac output are occurring. Various thresholds for stimulation that are applied by the stimulating devices may, in some embodiments, be determined based on such feedback. Thresholds may relate to the amplitude or frequency of electric or other stimulation. Thresholds may also be related to whether to initiate stimulation by the stimulating devices based on the feedback.

During and/or after stimulus is applied with the stimulating devices, the sensing devices may monitor the physiologic response of the subject. If stimulation is successful in achieving a desired response, the stimulation may be discontinued. Otherwise, the type, timing, etc., of stimulation may be adjusted.

In some embodiments, a user of the modular physiologic monitoring system may set preferences for the stimulus type, level, and/or otherwise personalize the sensation during a setup period or at any point during use of the modular physiologic monitoring system. The user of the modular physiologic monitoring system may be the subject being monitored and stimulated by the sensing devices and stimulating devices, or a doctor, nurse, physical therapist, medical assistant, caregiver, etc., of the subject being monitored and stimulated. The user may also have the option to disconnect or shut down the modular physiologic monitoring system at any time, such as via operation of a switch, pressure sensation, voice operated instruction, etc.

Stimulus or feedback which may be provided via one or more stimulating devices in a modular physiologic monitoring system may be in various forms, including physical stimulus (e.g., electrical, thermal, vibrational, pressure, stroking, a combination thereof, or the like), optical stimulus, acoustic stimulus, etc.

Physical stimulus may be provided in the form of negative feedback, such as in a brief electric shock or impulse as described above. Data or knowledge from waveforms applied in conducted electrical weapons (CEWs), such as in electroshock devices, may be utilized to avoid painful stimulus. Physical stimulus may also be provided in the form of positive feedback, such as in evoking pleasurable sensations by combining non-painful electrical stimulus with pleasant sounds, music, lighting, smells, etc. Physical stimulus is not limited solely to electrical shock or impulses. In other embodiments, physical stimulus may be provided by adjusting temperature or other stimuli, such as in providing a burst of cool or warm air, a burst of mist, vibration, tension, stretch, pressure, etc.

Feedback provided via physical stimulus as well as other stimulus described herein may be synchronized with, initiated by or otherwise coordinated or controlled in conjunction with one or more monitoring devices (e.g., a host device, one or more sensing devices, etc.). The monitoring devices may be connected to the stimulating devices physically (e.g., via one or more wires or other connectors), wirelessly (e.g., via radio or other wireless communication), etc. Physical stimulus may be applied to various regions of a subject, including but not limited to the wrist, soles of the feet, palms of the hands, nipples, forehead, ear, mastoid region, the skin of the subject, etc.

Optical stimulus may be provided via one or more stimulating devices. The optical stimulus may be positive or negative (e.g., by providing pleasant or unpleasant lighting or other visuals). Acoustic stimulus similarly may be provided via one or more stimulating devices, as positive or negative feedback (e.g., by providing pleasant or unpleasant sounds). Acoustic stimulus may take the form of spoken words, music, etc. Acoustic stimulus, in some embodiments may be provided via smart speakers or other electronic devices such as Amazon Echo®, Google Home®, Apple Home Pod®, etc. The stimulus itself may be provided so as to elicit a particular psychophysical or psychoacoustic effect in the subject, such as directing the subject to stop an action, to restart an action (such as breathing), to adjust an action (such as a timing between a step and a respiratory action, between a muscle contraction and a leg position, etc.).

As described above, the modular physiologic monitoring system may operate in a therapeutic mode, in that stimulation is provided when one or more cardiac parameters of a subject indicate some event (e.g., actual, imminent or predicted failure or worsening). The modular physiologic monitoring system, however, may also operate as or provide a type of cardiac “pacemaker” in other embodiments. In such embodiments, the modular physiologic monitoring system has the potential to reduce the frequency of cardiac events, or to possibly avoid certain cardiac events altogether. A modular physiologic monitoring system may provide functionality for timing and synchronizing periodic compression and relaxation of microvascular blood vessel networks with cardiac output. Such techniques may be utilized to respond to a type of failure event as indicated above. Alternatively or additionally, such techniques may be provided substantially continuously, so as to improve overall cardiac performance (e.g., blood flow) with the same or less cardiac work.

In some embodiments, a modular physiologic monitoring system may be configured to provide multi-modal stimuli to a subject. Multi-modal approaches use one or more forms of stimulation (e.g., thermal and electrical, mechanical and electrical, etc.) in order to mimic another stimulus to trick local nerves into responding in the same manner to the mimicked stimulus. In addition, in some embodiments multi-modal stimulus or input may be used to enhance a particular stimulus. For example, adding a mimicked electrical stimulus may enhance the effect of a thermal stimulus.

Modular physiologic monitoring systems may use pulses across space and time (e.g., frequency, pulse trains, relative amplitudes, etc.) to mimic vibration, comfort or discomfort, mild or greater pain, wet sensation, heat/cold, training neuroplasticity, taste (e.g., using a stimulating device placed in the mouth or on the tongue of a subject to mimic sour, sweet, salt, bitter or umami flavor), tension or stretching, sound or acoustics, sharp or dull pressure, light polarization (e.g., linear versus polar, the “Haidinger Brush”), light color or brightness, etc.

Stimulus amplification may also be provided by one or more modular physiologic monitoring systems using multi-modal input. Stimulus amplification represents a hybrid approach, wherein a first type of stimulus may be applied and a second, different type of stimulus provided to enhance the effect of the first type of stimulus. As an example, a first stimulus may be provided via a heating element, where the heating element is augmented by nearby electrodes or other stimulating devices that amplify and augment the heating stimulus using electrical mimicry in a pacing pattern. Electrical stimulus may also be used as a supplement or to mimic various other types of stimulus, including but not limited to vibration, heat, cold, etc. Different, possibly unique, stimulation patterns may be applied to the subject, with the central nervous system and peripheral nervous system interpreting such different or unique stimulation patterns as different stimulus modalities.

Another example of stimulus augmentation is sensing a “real” stimulus, measuring the stimulus, and constructing a proportional response by mimicry such as using electric pulsation. The real stimulus, such as sensing heat or cold from a Peltier device, may be measured by electrical-thermal conversion. This real stimulus may then be amplified using virtual mimicry, which may provide energy savings and the possibility of modifying virtual stimulus to modify the perception of the real stimulus.

In some embodiments, the stimulating devices in a modular physiologic monitoring system include an electrode array that attaches (e.g., via an adhesive or which is otherwise held in place) to a preferred body part. One or more of the stimulating devices may include a multiplicity of both sensing and stimulation electrodes, including different types of sensing and/or stimulation electrodes. The sensing electrodes on the stimulation devices, in some embodiments, may be distinct from the sensing devices in the modular physiologic monitoring system in that the sensing devices in the modular physiologic monitoring system may be used to measure physiologic parameters of the subject while the sensing electrodes on the stimulation devices in the modular physiologic monitoring system may be utilized to monitor the application of a stimulus to the subject.

A test stimulus may be initiated in a pattern in the electrode array, starting from application via one or a few of the stimulation electrodes and increasing in number over time to cover an entire or larger portion of the electrode array. The test stimulus may be used to determine the subject's response to the applied stimulation. Sensing electrodes on the stimulation devices may be used to monitor the application of the stimulus. The electrode array may also be used to record a desired output (e.g., physiologic parameters related to cardiac output). As such, one or more of the electrodes in the array may be configured so as to measure the local evoked response associated with the stimulus itself. Such an approach may be advantageous to confirm capture of the target nerves during use. By monitoring the neural response to the stimulus, the stimulus parameters including amplitude, duration, pulse number, etc., may be adjusted while ensuring that the target nerves are enlisted by the stimulus in use.

The test stimulus may migrate or be applied in a pattern to different electrodes at different locations in the electrode array. The response to the stimulus may be recorded or otherwise measured, using the sensing devices in the modular physiologic monitoring system and/or one or more of the sensing electrodes of the stimulating devices in the modular physiologic monitoring system. The response to the test stimulus may be recorded or analyzed to determine an optimal sensing or application site for the stimulus to achieve a desired effect or response in the subject. Thus, the test stimulus may be utilized to find an optimal sensing (e.g., dermatome driver) location. This allows for powerful localization for optimal pacing or other application of stimulus, which may be individualized for different subjects.

A stimulating device applied to the subject via an adhesive (e.g., an adhesively applied stimulating device), may be in the form of a disposable or reusable unit, such as a patch and/or patch-module or patch/hub pair as described above with respect to FIG. 1. An adhesively applied stimulating device, in some embodiments, includes a disposable interface configured so as to be thin, stretchable, able to conform to the skin of the subject, and sufficiently soft for comfortable wear. The disposable interface may be built from very thin, stretchable and/or breathable materials, such that the subject generally does not feel the device on his or her body.

The adhesively applied stimulating device also includes a means for interfacing with the subject through an adhesive interface and/or a window in the adhesive interface. Such means may include a plurality of electrodes that are coupled with a reusable component of the adhesively applied stimulating device and that are coupled to the body of the subject through the adhesive interface. The means may also or alternatively include: a vibrating actuator to provide vibration normal to and/or transverse to the surface of the skin on which the adhesively applied stimulating device is attached to the subject; a thermal device such as a Peltier device, a heating element, a cooling element, an RF heating circuit, an ultrasound source, etc.; a means for stroking the skin such as a shape memory actuator, an electroactive polymer actuator, etc.; a means for applying pressure to the skin such as a pneumatic actuator, a hydraulic actuator, etc.

Actuation means of the adhesively applied stimulating device may be applied over a small region of the applied area of the subject, such that the adhesive interface provides the biasing force necessary to counter the actuation of the actuation means against the skin of the subject.

Adhesively applied stimulating devices may be provided as two components—a disposable body interface and a reusable component. The disposable body interface may be applied so as to conform to the desired anatomy of the subject, and wrap around the body such that the reusable component may interface with the disposable component in a region that is open and free from a natural interface between the subject and another surface.

An adhesively applied stimulating device may also be a single component, rather than a two component or other multi-component arrangement. Such a device implemented as a single component may include an adhesive interface to the subject including two or more electrodes that are applied to the subject. Adhesively applied stimulating devices embodied as a single component provide potential advantages such as easier application to the body of the subject, but may come at a disadvantage with regards to one or more of breathability, conformity, access to challenging interfaces, etc., relative to two component or multi-component arrangements.

A non-contacting stimulating device may be, for example an audio and/or visual system, a heating or cooling system, etc. Smart speakers and smart televisions or other displays are examples of audio and/or visual non-contacting stimulation devices. A smart speaker, for example, may be used to provide audible stimulus to the subject in the form of an alert, a suggestion, a command, music, other sounds, etc. Other examples of non-contacting stimulating devices include means for controlling temperature such as fans, air conditioners, heaters, etc.

One or more stimulating devices may also be incorporated in other systems, such as stimulating devices integrated into a bed, chair, operating table, exercise equipment, etc., that a subject interfaces with. A bed, for example, may include one or more pneumatic actuators, vibration actuators, shakers, or the like to provide a stimulus to the subject in response to a command, feedback signal or control signal generated based on measurement of one or more physiologic parameters of the subject utilizing one or more sensing devices.

Although the disclosure has discussed devices attached to the body for monitoring aspects of the subject's disorder and/or physiologic information, as well as providing a stimulus, therapeutic stimulus, etc., alternative devices may be considered. Non-contacting devices may be used to obtain movement information, audible information, skin blood flow changes (e.g., such as by monitoring subtle skin tone changes which correlate with heart rate), respiration (e.g., audible sounds and movement related to respiration), and the like. Such non-contacting devices may be used in place of or to supplement an on-body system for the monitoring of certain conditions, for applying stimulus, etc. Information captured by non-contacting devices may, on its own or in combination with information gathered from sensing devices on the body, be used to direct the application of stimulus to the subject, via one or more stimulating devices on the body and/or via one or more non-contacting stimulating devices.

In some embodiments, aspects of monitoring the subject utilizing sensing devices in the modular physiologic monitoring system may utilize sensing devices that are affixed to or embodied within one or more contact surfaces, such as surfaces on a piece of furniture on which a subject is positioned (e.g., the surface of a bed, a recliner, a car seat, etc.). The surface may be equipped with one or more sensors to monitor the movement, respiration, HR, etc., of the subject. To achieve reliable recordings, it is advantageous to have such surfaces be well positioned against the subject. It is also advantageous to build such surfaces to take into account comfort level of the subject to keep the subject from feeling the sensing surfaces and to maintain use of the sensing surface over time.

Stimulating devices, as discussed above, may take the form of audio, visual or audiovisual systems or devices in the sleep space of the subject. Examples of such stimulating devices include smart speakers. Such stimulating devices provide a means for instruction a subject to alter the sleep state thereof. The input or stimulus may take the form of a message, suggestion, command, audible alert, musical input, change in musical input, a visual alert, one or more lights, a combination of light and sound, etc. Examples of such non-contacting stimulating devices include systems such as Amazon Echo®, Google Home®, Apple Home Pod®, and the like.

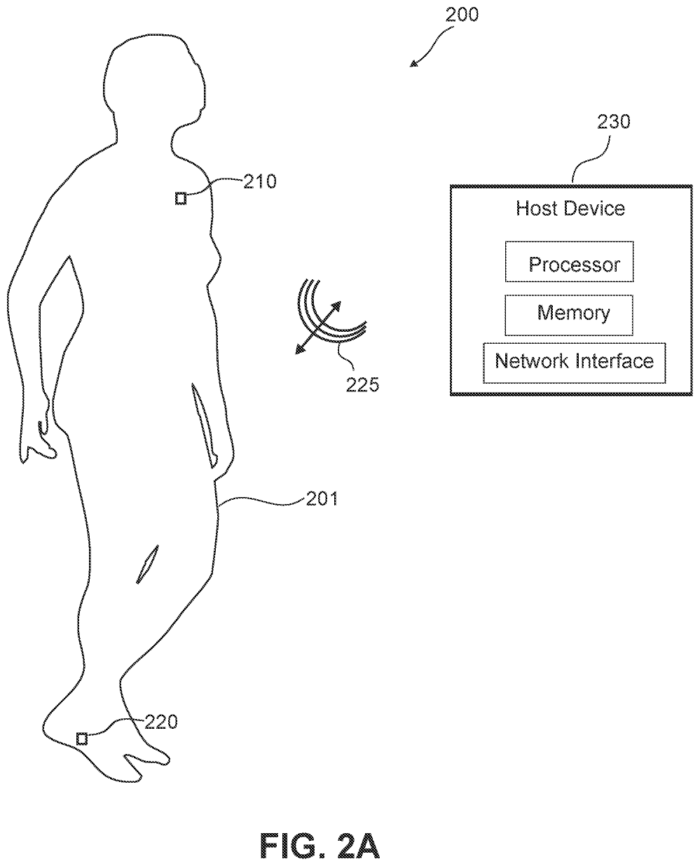

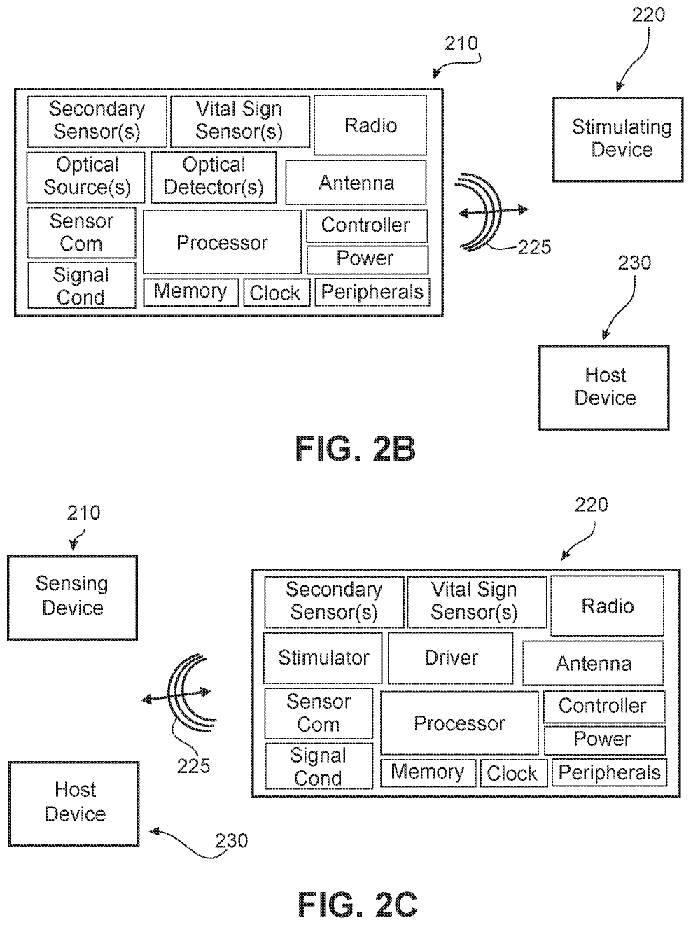

FIGS. 2A-2C show a modular physiologic monitoring system 200. The modular physiologic monitoring system 200 includes a sensing device 210 and a stimulating device 220 attached to a subject 201 that are in wireless communication 225 with a host device 230. The host device 230 includes a processor, a memory and a network interface.

The processor may comprise a microprocessor, a microcontroller, an application-specific integrated circuit (ASIC), a field-programmable gate array (FPGA) or other type of processing circuitry, as well as portions or combinations of such circuitry elements.

The memory may comprise random access memory (RAM), read-only memory (ROM) or other types of memory, in any combination. The memory and other memories disclosed herein may be viewed as examples of what are more generally referred to as “processor-readable storage media” storing executable computer program code or other types of software programs. Articles of manufacture comprising such processor-readable storage media are considered embodiments of the invention. A given such article of manufacture may comprise, for example, a storage device such as a storage disk, a storage array or an integrated circuit containing memory. The processor may load the computer program code from the memory and execute the code to provide the functionalities of the host device 230.

The network interface provides circuitry enabling wireless communication between the host device 230, the sensing device 210 and the stimulating device 220.

FIG. 2A illustrates a modular physiologic monitoring system 200 that includes only a single instance of the sensing device 210 and the stimulating device 220 for clarity. It is to be appreciated, however, that modular physiologic monitoring system 200 may include multiple sensing devices and/or multiple stimulating devices. In addition, although FIG. 2A illustrates a modular physiologic monitoring system 200 in which the sensing device 210 and the stimulating device 220 are attached to the subject 201, embodiments are not limited to such arrangements. As described above, one or more sensing and/or stimulating devices may be part of contacting surfaces or non-contacting devices. In addition, the placement of sensing device 210 and stimulating device 220 on the subject 201 may vary as described above. Also, the host device 230 may be worn by the subject 201, such as being incorporated into a smartwatch or other wearable computing device. The functionality provided by host device 230 may also be provided, in some embodiments, by one or more of the sensing device 210 and the stimulating device 220. In some embodiments, as will be described in further detail below, the functionality of the host device 230 may be provided at least in part using cloud computing resources.

FIG. 2B shows a schematic diagram of aspects of the sensing device 210 in modular physiologic monitoring system 200. The sensing device 210 includes one or more of a processor, a memory device, a controller, a power supply, a power management and/or energy harvesting circuit, one or more peripherals, a clock, an antenna, a radio, a signal conditioning circuit, optical source(s), optical detector(s), a sensor communication circuit, vital sign sensor(s), and secondary sensor(s). The sensing device 210 is configured for wireless communication 225 with the stimulating device 220 and host device 230.

FIG. 2C shows a schematic diagram of aspects of the stimulating device 220 in modular physiologic monitoring system 200. The stimulating device 220 includes one or more of a processor, a memory device, a controller, a power supply, a power management and/or energy harvesting circuit, one or more peripherals, a clock, an antenna, a radio, a signal conditioning circuit, a driver, a stimulator, vital sign sensor(s), a sensor communication circuit, and secondary sensor(s). The stimulating device 220 is configured for wireless communication 225 with the sensing device 210 and host device 230.

Communication of data from the sensing devices and/or stimulating devices (e.g., patches and/or patch-module pairs) may be performed via a local personal communication device (PCD). Such communication in some embodiments takes place in two parts: (1) local communication between a patch and/or patch-module pair (e.g., via a hub or module of a patch-module pair) and the PCD; and (2) remote communication from the PCD to a back-end server, which may be part of a cloud computing platform and implemented using one or more virtual machines (VMs) and/or software containers. The PCD and back-end server may collectively provide functionality of the host device as described elsewhere herein.

Illustrative embodiments provide systems, devices and methods for noninvasive detection of hemorrhage and shock.

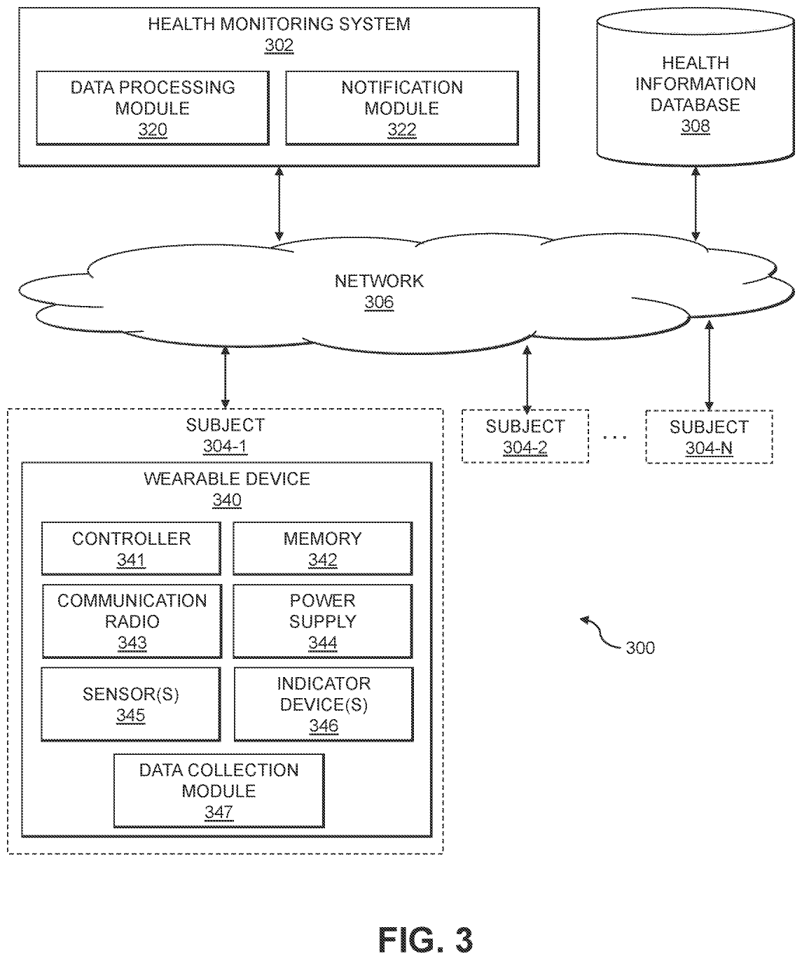

FIG. 3 shows a system 300 configured for noninvasive detection of hemorrhage and shock, which includes a health monitoring system 302 and subjects 304-1, 304-2, . . . 304-N (collectively, subjects 304). The health monitoring system 302 is configured to monitor the subjects 304, so as to detect various health conditions (e.g., hemorrhage and shock). To do so, the health monitoring system 302 obtains information from devices associated with the subjects 304 over network 306. For example, FIG. 3 shows subject 304-1 associated with a wearable device 340. Although not shown in FIG. 3 for clarity of illustration, other ones of the subjects 304-2 through 304-N may be associated with wearable devices configured in a manner similar to that described herein with respect to wearable device 340.

The wearable device 340 includes a controller 341, a memory 342, a communication radio 343, a power supply 344, one or more sensors 345, one or more indicator devices 346, and a data collection module 347. The data collection module 347 is configured to collect physiologic data associated with the subject 304-1 from at least one of the one or more sensors 345, and to communicate such physiologic data or physiologic metrics derived therefrom to the health monitoring system 302 over network 306 using the communication radio 343 (e.g., a network interface). In some embodiments, the data collection module 347 of the wearable device 304 may communicate the physiologic data for storage in a health information database 308 also coupled to the network 306, rather than or in addition to communicating the physiologic data to the health monitoring system 302.

The physiologic data obtained from the sensors 345 may include one or more physiologic and/or electrophysiological signals captured from the subject 304-1. The sensors 345 may be embodied as one or more patch-module pairs or patch interfaces as described above in conjunction with FIGS. 1 and 2A-2C. Such patch-module pairs or patch interfaces embodying the sensors 345 may be configured for attachment to the subject 304-1, and may be configured to convey and/or store one or more physiologic, electrophysiological, and/or physical signals, or signals or metrics derived therefrom (e.g., for temporary storage in memory 342 of the wearable device 340 prior to sending such data to the health monitoring system 302 and/or health information database 308).

In some embodiments, the wearable device 340 comprises or represents a host device as described above in conjunction with FIGS. 1 and 2A-2C, where the host device is coupled in wireless communication or physical communication with the sensors 345 (e.g., embodied as patch-module pairs, or more generally sensing devices). This coupling may be as part of a body area network (BAN). The wearable device 340 may include features for recharging one or more of the sensors 345 (e.g., using power supply 344), for performing diagnostic tests on one or more of the sensors 345, etc.

The wearable device 340 may include a plurality of sensors 345, with individual ones of the sensors 345 being hot swappable so as to maintain a continuous or nearly continuous monitoring of the subject 304-1.

The controller 341 of the wearable device 340 may comprise a central processing unit (CPU) for carrying out instructions of one or more computer programs for performing arithmetic, logic, control and input/output (I/O) operations specified by the instructions. Such computer programs may be stored in memory 342. The memory 342 provides electronic circuitry configured to temporarily store data that is utilized by the controller 341. In some embodiments, the memory 342 further provides persistent storage for storing data utilized by the controller 341.

The communication radio 343 is a network controller or interface configured for physical or wireless connection of the wearable device 340 to one or more networks. For example, the communication radio 343 may be used to connect the wearable device 340 to network 306 for exchanging data with the health monitoring system 302, the health information database 308, etc. The communication radio 343 may further or alternatively be used for connecting to a BAN including various sensing devices (e.g., sensors 345), the one or more indicator devices 346, etc. The communication radio 343 may comprise an antenna and a network interface controller.

The network 306 may comprise a physical connection (wired or wireless), the Internet, a cloud communication network, etc. Examples of wireless communication networks that may be utilized include networks that utilize Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

The power supply 344 provides a source of electrical current for powering the wearable device 340, for charging sensing devices comprising the sensors 345 or the indicator devices 346, etc. In some embodiments, the power supply 344 is a battery that is consumable or rechargeable, and may be user-replaceable or permanently installed in the wearable device 340. The power supply 344 may alternatively be an external power source that is permanently or removably connected to the wearable device 340. In some embodiments, the power supply 344 is a removable external power source that is wirelessly connected using a pair of induction coils to transfer electricity from a source coil to a coil installed within the wearable device 340.

The sensors 345, in some embodiments, are non-invasive measurement tools for monitoring one or more characteristics or metrics associated with the subject 304-1. One or more of the sensors 345 may be configured to interface with the subject 304-1. The sensors 345 may include one or more of: an electrophysiologic sensor, a temperature sensor, a thermal gradient sensor, a barometer, an altimeter, an accelerometer, a gyroscope, a humidity sensor, a magnetometer, an inclinometer, an oximeter, a colorimetric monitor, a sweat analyte sensor, a galvanic skin response sensor, an interfacial pressure sensor, a flow sensor, a stretch sensor, a microphone, combinations thereof, etc.

In some embodiments, one or more of the sensors 345 configured for monitoring one or more physiologic and/or electrophysiological signals from the subject 304-1 comprise a soft, breathable and hypoallergenic device that may be affixed to one or more sites on the subject 304-1 to obtain one or more local physiologic and/or electrophysiologic signals. One or more of the sensors 345 may comprise a conducting adhesive (e.g., an anisotropically conducting adhesive, with the direction of conduction being oriented substantially normal to the surfaces of a substrate of the sensors 345).

One or more of the sensors 345 may also or alternatively include an orientation sensor configured to obtain an orientation signal, where the controller 341 is configured to receive the orientation signal or a signal generated therefrom, and to incorporate the orientation signal into the analysis. Some non-limiting examples of orientation sensors include one or more of an altimeter, a barometer, a tilt sensor, a gyroscope, combinations thereof, etc.

In some embodiments, one or more of the sensors 345 include a temperature sensor configured to generate a temperature signal from the subject 304-1 or a signal generated therefrom, where the controller 341 is configured to receive the temperature signal and to assess a thermal state of the subject 304-1 therefrom.

One or more of the sensors 345 may comprise or provide an EEG device including a hydration sensor configured to generate a fluid level signal from the subject 304-1, where the controller 341 is configured to receive the fluid level signal or a signal generated therefrom, and to assess the hydration state of the subject 304-1 therefrom.

In some embodiments, one or more of the sensors 345 are configured to assess a sleep state of a subject and include an EMG/EOG device (e.g., one or more patch-module pairs as described above configured to measure local EMG and/or EOG signals from adjacent tissues). The EMG/EOG device may be configured to measure one or more EMG and/or EOG signals from the head or neck of the subject 304-1. The controller 341, or another type of processor included in or coupled to the EMG/EOG device, is configured to receive the EMG/EOG signal, or signals generated therefrom, and to analyze such signals to determine the sleep state of the subject 304-1. The EMG/EOG device may include a microphone configured to obtain an acoustic signal from the subject 304-1, where the controller 341 is configured to receive the acoustic signal or a signal generated therefrom, and to incorporate the acoustic signal into the assessment.

The sensors 345 may also include a plurality of EMG devices in some embodiments, where each EMG device is configured to monitor a separate muscle group on the subject 304-1. The controller 341 is configured to synchronize and analyze the EMG signals received from each of at least a subset of the plurality of EMG devices to determine one or more characteristics of the subject 304-1, such as at least a portion of the gait and/or muscle movement of the subject 304-1. One or more of the plurality of EMG devices may include one or more orientation, kinetic, kinematic and/or proprioception sensors configured so as to generate one or more kinematic signals. The controller 341 is configured to incorporate the kinematic signal into the analysis. The controller 341 may also or alternatively be configured to analyze one or more of the EMG signals to generate one or more muscle exertion metrics.

One or more of the sensors 345 may include sensors for evaluating SpO2 at one or more sites on the subject 304-1 to obtain an oxygen saturation signal from the subject 304-1. The controller 341 is configured to receive the oxygen saturation signal or one or more signals or metrics generated therefrom, and to incorporate the oxygen saturation signal into the assessment.

The indicator devices 346 may include various types of devices for delivering notifications to the subject 304-1 (or to a doctor, nurse, physical therapist, medical assistant, caregiver, etc. associated with the subject 304-1). In some embodiments, one or more of the indicator devices 346 comprise one or more light emitting diodes (LEDs), a liquid crystal display (LCD), a buzzer, a speaker, a bell, etc., for delivering one or more visible or audible notifications. More generally, the indicator devices 346 may include any type of stimulating device as described herein which may be used to deliver notifications to the subject 304-1 (or to a doctor, nurse, physical therapist, medical assistant, caregiver, etc., associated with the subject 304-1).

The data collection module 347, as will be described in further detail below, is configured to poll at least one of the one or more sensors 345 and to store collected data in the memory 342. The collected data may also be provided over network 306 (e.g., using communication radio 343) for storage in the health information database 308.

The health monitoring system 302, in some embodiments, is implemented as an application or applications running on one or more physical or virtual computing resources. Physical computing resources include, but are not limited to, a smartphone, laptop, tablet, desktop, wearable computing device, server, etc. Virtual computing resources include, but are not limited to, VMs, software containers, etc. The physical and/or virtual computing resources implementing the health monitoring system 302, in some embodiments, may be part of a cloud computing platform. A cloud computing platform includes one or more clouds providing a scalable network of computing resources (e.g., including one or more servers and databases). In some embodiments, the clouds of the cloud computing platform implementing the health monitoring system 302 are accessible via the Internet. In other embodiments, the clouds of the cloud computing platform implementing the health monitoring system 302 may be private clouds where access is restricted (e.g., such as to one or more credentialed medical professionals or other authorized users). In these and other embodiments, the health monitoring system 302 may be considered as forming part of an emergency health network comprising at least one server and at least one database (e.g., the health information database 308) storing health data pertaining to one or more patients (e.g., one or more of the subjects 304).

The health information database 308 provides a database configured for storing information about patient conditions. For example, the health information database 308 may store information collected by the data collection module 347 of wearable device 340 associated with subject 304-1, which polls one or more of the sensors 345 that are part of or otherwise in communication with the wearable device 340. As shown in FIG. 3, the health information database 308 is located on or accessible via network 306 and is implemented external to the health monitoring system 302. In other embodiments, however, the health information database 308 may be implemented at least in part internal to the health monitoring system 302. The health information database 308, for example, may be implemented as part of the same cloud computing platform that implements the health monitoring system 302. The health information database 308 may also be located at least in part within the memory 342 of the wearable device 340.

The health monitoring system 302, as shown in FIG. 3, includes a data processing module 320 and a notification module 322. The data processing module 320 obtains sensor data from the health information database 308 (or directly from wearable devices associated with the subjects 304, such as from wearable device 340 associated with subject 304-1). The data processing module 320 is configured to perform various operations on the sensor data, such as one or more of analysis, amplification, comparison to a baseline sample, comparison to a predictive model, etc., to identify whether the sensor data is indicative of a subject in a pre-shock or hemorrhagic state. For example, where the sensor data is collected from the wearable device 340 (e.g., either directly, or from the health information database 308), the data collection module 320 may identify that the subject 304-1 is in a pre-shock state or hemorrhagic state. In response, the data processing module 320 may trigger the notification module 322.

The notification module 322 is configured to determine one or more notification settings associated with the subject 304-1, and to execute or deliver notifications in accordance with the determined notification settings. The notification settings, in some embodiments, may specify the types of indicator devices 346 that are part of or otherwise accessible to the wearable device 340 for delivering notifications to the subject 304-1 (or to a doctor, nurse, physical therapist, medical assistant, caregiver, etc., associated with the subject 304-1). As noted above, the indicator devices 346 in some embodiments may be configured to deliver visual or audible alarms. In other embodiments, the indicator devices 346 may be configured to provide stimulus or feedback via stimulating devices associated with the subject 304-1. Such stimulus or feedback, as detailed above, may include physical stimulus (e.g., electrical, thermal, vibrational, pressure, stroking, a combination thereof, or the like), optical stimulus, acoustic stimulus, etc. In some embodiments, the notification module 322 may be further or alternatively configured to deliver notifications to remote terminals or devices other than the wearable device 340 associated with subject 304-1. For example, notifications may be delivered to one or more devices associated with a doctor, nurse, physical therapist, medical assistant, caregiver, etc., associated with the subject 304-1. The notification module 322 may also or alternatively execute the notifications via some predefined protocols as specified by the determined notification settings.

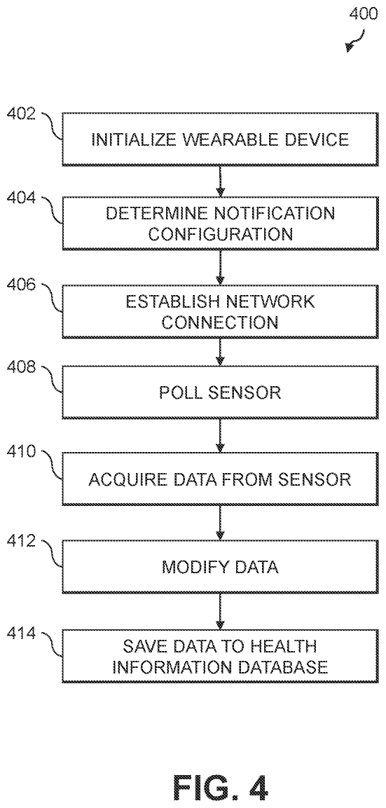

Functioning of the data collection module 347 of the wearable device 340 will now be described in further detail with respect to the process flow 400 of FIG. 4. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process flow 400 of FIG. 4 begins in step 402 with initializing the wearable device 340. Step 402 may include powering on or otherwise activating the wearable device 340 (e.g., waking from a sleep or suspend state) that is associated with subject 304-1 (e.g., affixed to the subject 304-1). In some embodiments, the wearable device 340, or one or more components thereof such as one or more of the sensors 345, is affixed proximal to a carotid artery of the subject 304-1. The wearable device 340 may comprise one or more physically distinct devices. In some embodiments, all components of the wearable device 340 are embodied within a single physical unit or form factor. In other embodiments, however, different components of the wearable device 340 are part of distinct physical units or form factors. As one example, components of the wearable device 340 such as the controller 341, memory 342, communication radio 343, power supply 344 and data collection module 347 may be part of one physical unit or form factor, while the sensors 345 (e.g., sensing devices) and indicator devices 346 (e.g., stimulating devices) are part of one or more additional physical units or form factors. In some embodiments, one or more of the sensors 345 and indicator devices 346 may be part of the same physical unit or form factor (e.g., such as a patch-module pair configured to provide or function as both a sensing and a stimulating device as described elsewhere herein). Step 402 may thus include the controller 341 of the wearable device 340 powering on or activating, waking or otherwise initializing one or more of the sensors 345 and indicator devices 346 which may be physically distinct or separate but in communication with a base computing unit provided by the controller 341.

In step 404, a notification configuration of the wearable device 340 is determined. Step 404 may include identifying the method or means of delivering one or more notifications in response to the data processing module 320 detecting that a trigger condition has occurred. The method or means of delivering the notification may be varied as described above. In some embodiments, the notification delivery method includes a desired type of visual or audible read-out or alert from the wearable device 340 (e.g., using one or more of the indicator devices 346).

The notification delivery method may also or alternatively comprise a visual or audible read-out or alert from a “local” device that is in communication with the wearable device 340. The local device may comprise, for example, a mobile computing device such as a smartphone, tablet, laptop etc., or another computing device, that is associated with the subject 304-1. A local device may also include devices connected to the wearable device 340 via a BAN or other type of local or short-range wireless network (e.g., a Bluetooth network connection).

The notification delivery method may further or alternatively comprise a visual or audible read-out or alert from a “remote” device that is in communication with the wearable device 340 or health monitoring system 302 via network 306. The remote device may be a mobile computing device such as a smartphone, tablet, laptop, etc., or another computing device (e.g., a telemetry center or unit within a hospital or other facility), that is associated with a doctor, nurse, physical therapist, medical assistant, caregiver, etc., monitoring the subject 304-1. It should be understood that the term “remote” in this context does not necessarily indicate any particular physical distance from the subject 304-1. For example, a remote device to which notifications are delivered may be in the same room as the subject 304-1. The term “remote” in this context is instead used to distinguish from “local” devices (e.g., in that a “local” device in some embodiments is assumed to be owned by, under the control of, or otherwise associated with the subject 304-1, while a “remote” device is assumed to be owned by, under the control of, or otherwise associated with a user other than the subject 304-1 such as a doctor, nurse, physical therapist, medical assistance, caregiver, etc.).

In step 406, a network connection is established with the device or devices to which the notifications are to be delivered. If the method of notification delivery is to a remote device (e.g., over a long-range network), step 406 may include establishing a connection with a wired or wireless access point such as a router, modem, or cell-based mobile network entity such as a base station in a 3G, LTE, 4G, 5G or another type of cellular network, etc. If the method of notification delivery is via a local device (e.g., over a short-range network rather than a long-rage network), step 406 may include establishing a connection via wired or wireless connection such as WiFi, Bluetooth, near-field communication (NFC), etc. If the method of notification delivery is only via the wearable device 340 (e.g., and not to a remote device or a local device separate from the wearable device 340), step 406 may include activating (e.g., powering, on, waking from a sleep or suspended state, etc.) one or more of the indicator devices 346 to be used for delivering the notifications. In embodiments where the method of delivery includes combinations of the above (e.g., delivery to one or more local devices, one or more remote devices, and/or one or via the wearable device 340), step 406 may include establishing multiple connections. For example, if the method of delivery for notifications includes delivery to a local and a remote device, step 406 may include establishing a first connection (e.g., a WiFi connection) with the local device and a second connection (e.g., a 4G cellular network connection) with the remote device.

At least one of the sensors 345 in communication with the wearable device 340 is polled in step 408. As described above and elsewhere herein, the sensors 345 may be various types including but not limited to electrocardiograph, ultrasonic transducers, plethysmograph, sphygmomanometer, glucometer, thermometer, capnograph, means of measuring electrodermal activity such as somatosensory response or dermal impedance response, etc. In the description below, various embodiments are described with respect to electrocardiograph sensors measuring a pulse waveform from a carotid artery. It should be appreciated, however, that various other types of sensors and associated signal measurements and metrics may be used in other embodiments in combination with or as an alternative to electrocardiograph sensors.

In step 410, data is acquired from the at least one sensor 345 in communication with the wearable device 340. In some embodiments, the at least one sensor 345 includes an EKG device placed onto a torso of the subject 304-1, the EKG device being configured to measure an electrocardiographic signal from the torso of the subject 304-1 so as to produce an EKG signal. The at least one sensor 345 may also include one or more pulse devices configured to measure local blood flow in adjacent tissues configured for placement onto one or more sites on one or more extremities of the subject 304-1, each of the pulse devices configured to measure a local pulse at the placement site so as to produce one or more pulse signals. The EKG devices and pulse devices, or more generally the sensors 345, may include one or more processors (e.g., illustrated in FIG. 3 as controller 341) configured to receive the EKG and pulse signals, or signals or metrics generated therefrom.

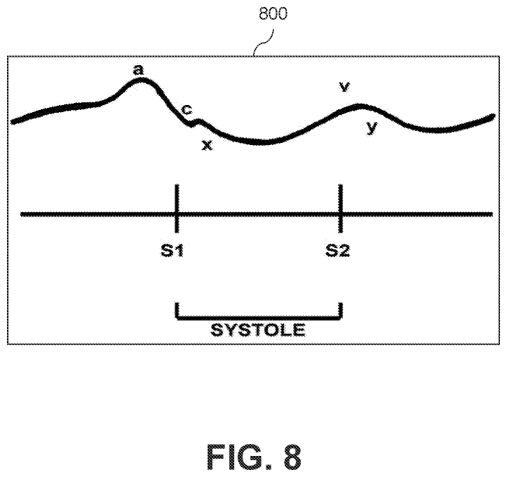

Various modifications to the acquired data may be performed in step 412. Such modification, in some embodiments, includes one or more of amplification, detection of a pattern or conversion of the data into a normalized format, etc. In some embodiments, modification of the data in step 412 includes segmentation of a pulse signal into repeatable waveforms as depicted in plot 800 of FIG. 8. The modification may further include averaging corresponding points on the waveforms so as to average their values and remove noise from the data, such as by identifying the arterial pressure maximum of the upstroke interval (e.g., denoted “a” in plot 800 of FIG. 8) and the notch on the descending phase of the upstroke interval (e.g., denoted “c” in plot 800 of FIG. 8). The temporal difference from the beginning of the waveform to each of the points may be averaged, along with averaging the magnitude of each of these points across a number “n” of waveforms representing a time interval “t.” Step 412 may further include calculating waveform or heart rate variability. It should be noted that step 412 is an optional step, and that in some embodiments the data acquired from the sensors 345 in step 410 is not modified. The collected data, with or without modification following step 412, may be saved to the health information database 308 in step 414.

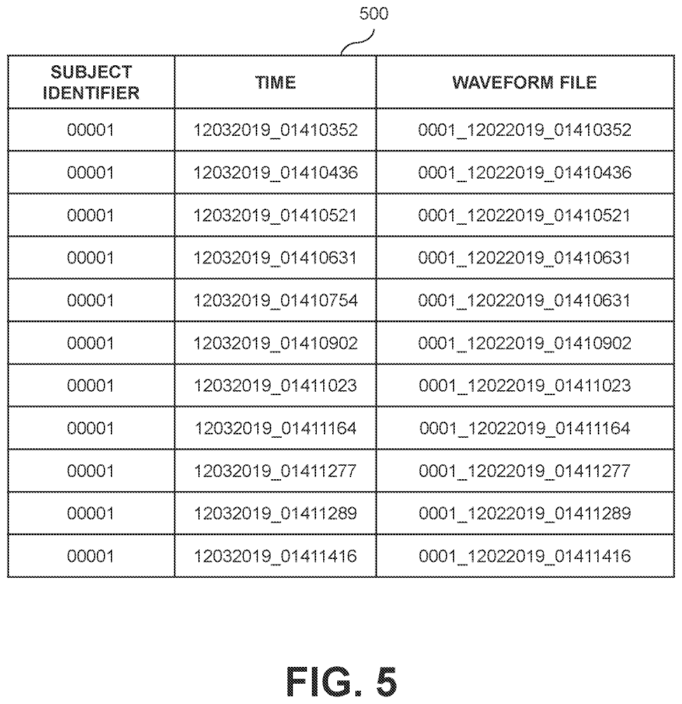

Aspects of the health information database 308 will now be described in further detail with respect to FIG. 5, show shows a table 500 illustrating information that may be stored in the health information database 308. The health information database 308 is configured to store information about patient conditions (e.g., of one or more of the subjects 304) or health state. Such information may include the data acquired from the sensors 345 using the data collection module 347 of wearable device as described above in conjunction with FIG. 4. The health information database 308 may be located on or accessible via network 306, such as being implemented using a cloud computing platform. In other embodiments, the health information database 308 may be maintained internal to the wearable device 340, such as in memory 342 thereof, or may be part of the health monitoring system 302, various combinations thereof, etc. FIG. 5 shows a table 500 illustrating information stored in the health information database 308, which may include images of pulse waveforms (e.g., such as the example shown in FIG. 8 described in further detail below), the times at which the waveforms were collected, and the patient or subject identifier of the subject (e.g., subject 304-1) that the waveforms were collected from. Although the table 500 in FIG. 5 shows example entries that are all obtained from the same patient or subject (e.g., subject 304-1 with associated subject identifier “00001”), in other embodiments the health information database 308 may store data that is collected from multiple ones of the subjects 304.

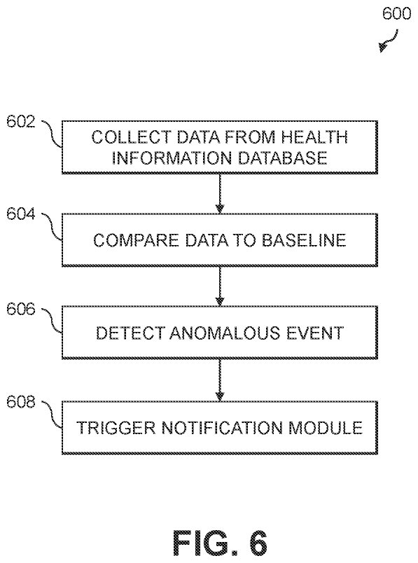

Functioning of the data processing module 320 of health monitoring system 302 will now be described in further detail with respect to the process flow 600 of FIG. 6. The process flow 600 begins in step 602 with collecting data pertaining to a particular patient or subject (e.g., subject 304-1). The data may be collected or obtained from the health information database 308, directly from the wearable device 340 associated with the subject 304-1, combinations thereof (e.g., obtaining older data from the health information database 308 and obtaining more recent data directly from the wearable device 340, etc.). When obtaining the data from the health information database 308, the data processing module 320 may be configured to submit a query to the health information database 308 with a patient or subject identifier of the subject 304-1 (e.g., “00001” in the example table 500 shown in FIG. 5). The data collected in step 602 may be varied, and may include data relating to various physiologic metrics or characteristics of the subject 304-1, such as heart rate, heart rate variability, amplitude and contour of one or more electrocardiograph waveforms, electrodermal activity, pulse volume, etc. In some embodiments, the collected data may include echoed signals from an ultrasound transducer (e.g., one of the sensors 345) integrated into the wearable device 340. The collected data may include one or more time series pulse waveforms from an electrocardiograph.

In step 604, the data collected in step 602 may be compared to baseline data. The baseline data may include prior data that is taken from the same patient or subject 304-1. The baseline data may further or alternatively include a data model that is derived from data collected from multiple patients in varying states of health (e.g., the subjects 304 collectively). The varying states of health, in some cases, include one or more states of hemorrhagic or hypovolemic shock. As noted above, the data collected in step 602 may include a time series pulse waveform (e.g., such as that illustrated in FIG. 8). In such embodiments, step 604 may include comparing the time series pulse waveform to an average adjusted pulse waveform (e.g., from the first minute of time series data collected during a current monitoring session).

The process flow 600 continues with step 606, detecting one or more anomalous events based at least in part on the step 604 comparison. Step 606 may include identifying at least one metric indicating a likelihood of an anomalous event, and calculating a probability that the anomalous event will occur. The at least one metric may include one or more of heart rate, heart rate variability, amplitude and contour of one or more electrocardiograph waveforms, etc. In some embodiments, the anomalous event is the occurrence of hemorrhagic and hypovolemic shock, and the metric is a decrease in amplitude of the pulse waveform from the electrocardiograph. Step 606 may utilize one or more machine learning algorithms (e.g., an a priori classifier, a random forest classifier, a decision tree learning algorithm, a structured prediction algorithm, a Bayesian network, a random field algorithm, a recurrent neural network (RNN), a convolutional neural network (CNN), etc.) in calculating a probability that the anomalous event will occur.

The notification module 322 of the health monitoring system 302 is triggered in step 608 if the probability of the anomalous event occurring exceeds some designated threshold value. The notification module 322, as will be described in further detail below, receives the detected anomalous event and the probability of the anomalous event occurring to determine actions to take (e.g., whether or not one or more notifications should be generated, where the one or more notifications should be delivered, etc.), the information to include while executing the action (e.g., the information to include in a generated notification, which may include information regarding why the notification was generated, steps to take so as to treat the anomalous event, etc.). The notification module 322 will further execute the action (e.g., to deliver the one or more generated notifications, etc.).

In some embodiments, the designated threshold value used in step 608 may be 50%, such that if the probability of an anomalous event occurring exceeds 50%, the notification module 322 is triggered. It should be appreciated, however, that various other thresholds may be used in other embodiments. Further, the probability of the anomalous event may be computed or otherwise determined in step 606 individually for two or more groups of metrics. For example, a first metric may be the decrease in amplitude of the pulse waveform from the electrocardiograph and a second metric may be related to one or more of heart rate and heart rate variability. The first metric may be associated with a first probability of the anomalous event occurring, and the second metric may be associated with a second probability of the anomalous event occurring. The first probability and the second probability may be combined (e.g., according to some designated weighting), or may be compared to individual thresholds. The notification module 322 may be triggered in step 408 if one of the first and second metric has an associated probability exceeding its associated threshold, if both of the first and second metric have associated probabilities exceeding their associated thresholds, etc.

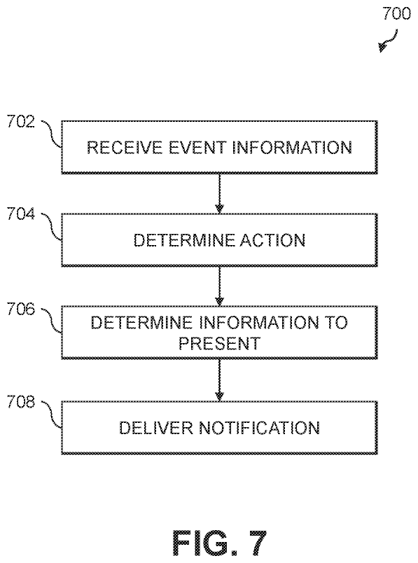

Functioning of the notification module 322 will now be described in further detail with respect to the process flow 700 of FIG. 7. In step 702, the notification module 322 receives event information from the data processing module 320. The event information may include the type of anomalous event detected, the probability of the anomalous event occurring, etc. Consider, as an example, a detected anomalous event of hypovolemic shock with an associated probability of occurrent of 74%.

Based upon the type of anomalous event detected and the probability of the anomalous event occurring, an action to take is determined in step 704. This may include looking up delivery notification settings for the type of anomalous event detected. Step 704 may also include obtaining information regarding notification settings that are particular to the particular patient or subject (e.g., subject 304-1) that is being analyzed.

Different ones of the subjects 304 may be associated with different notification settings for different types of anomalous events. For example, a first subject (e.g., 304-1) may be associated with first notification settings indicating that notifications should only be delivered if the probability of a given anomalous event is greater than a first designated threshold, while a second subject (e.g., 304-2) may be associated with second notification settings indicating that notifications should only be delivered if the probability of the given anomalous event is greater than a second designated threshold different than the first designated threshold.

Various other examples are possible, including where the notification settings indicate that different types and numbers of notifications should be generated and delivered based on the probability of the given anomalous event occurring. For example, the first notification settings for the first subject 304-1 may indicate that a first type of notification is generated for delivery to the subject 304-1 when the probability of the given anomalous event occurring is greater than the first designated threshold, that a second type of notification is generated for delivery to the subject 304-1 when the probability of the given anomalous event occurring is greater than a third designated threshold, etc. The notification settings may also indicate where the notifications should be delivered, such as to one or more local devices, one or more remote devices, combinations thereof, etc.

Continuing with the example above, assume that the anomalous event is indicative of hypovolemic shock with a probability of the event occurring being 74%. The notification settings may indicate for this combination of anomalous event type and probability, the method of notification delivery is via transmission to a local device (e.g., to one or more indicator devices 346 in communication with wearable device 340 associated with subject 304-1). The notification settings may further indicate that, because the probability of the event occurring is above 50%, a visual alert should be displayed on the local device, but because the probability is less than 75%, an audible alert should not be delivered.

In step 706, a determination is made as to which information is to be presented via the generated notifications. The information to present may be based on the type of anomalous event detected, the probability of the anomalous event occurring, notification settings of the associated subject, etc. Continuing again with the example above, the pulse waveform data may indicate a likelihood of hypovolemic shock. In this instance, the information that may be most useful to a rescuer (e.g., an emergency medical technician (EMT), doctor, nurse, other caregiver, etc.) is the subject's heart rate and blood pressure. Therefore, step 706 may include determining that heart rate and blood pressure data should be delivered as part of the generated notifications if such data is available.

The generated notifications are delivered in step 708, with the delivered notifications include the information determined in step 706. Continuing again with the example above, a visual alert may be delivered to a local device indicating a high likelihood of hypovolemic shock, where the visual alert includes the subject's heart rate and blood pressure.

FIG. 8, as noted above, shows an image 800 of a pulse waveform. The pulse waveform is representative of arterial pressure measured over one heartbeat. In the image 800, “a” identifies the arterial pressure maximum of the upstroke interval, “c” identifies the notch on the descending phase of the upstroke interval, “x” denotes a pressure descent representing atrial relaxation, “v” denotes a peak representing atrial filling, and “y” denotes a pressure descent corresponding to the beginning of diastole.

Early hemorrhage and shock is not easily detected using noninvasive technology. The ability to noninvasively obtain physiologic parameters suggesting early or imminent shock has substantial applications in emergency medicine, medical aspects of military combat for triage, treatment, prognosis, etc. In illustrative embodiments, devices (e.g., wearable device 340 in FIG. 3, patch-module pairs shown and described above with respect to FIGS. 1 and 2A-2C, etc.) are used to obtain signals from the vasculature of a subject that will indicate health or various disease states. Such devices may be used to detect, transduce, amplify, process, record, transmit, and compare all data obtained. Two-way communication with the devices may be used. For example, the devices may be interrogated from remote locations, and can be controlled and programmed from remote locations.

In some embodiments, the devices include wearable devices that are attached to the skin of a subject over important vascular structures, especially in the neck (e.g., both arteries and veins). The devices are configured to detect, amplify, process, record and transmit data. The data may include high or low frequency sonic waveform energy in a “chirp” mode (e.g., in a range of 100 kilohertz (kHz) to 20 megahertz (MHz)). The devices may detect various physiologic signals and associated metrics, such as: heart rate/pulse rate; amplitude and contour; pulse waves (along with contour); timing of carotid changes, such as in relationship to S1, S3, S3 heart sounds; etc. These and other collected data may be used to detect various abnormalities, including pulses alternan, pulses paradoxus, bruits/thrills (e.g., high-frequency mechanical information), etc.

Devices in some embodiments may be used to detect variability in heart rate and/or pulse for estimation of autonomic tone, to detect pulse volume (e.g., including correlation with other parameters), to detect pulse contour (e.g., weak pulse and trends in pulse upstroke, downstroke, dwell, etc.), etc. The collected data may be compared over time looking for trends determining health, disease, or impending minor or severe health problems related to central hemodynamics and physiology, etc.

Consider, as an example, the carotid artery. Abnormalities of carotid pulse may involve alteration in the amplitude of the pulse peak, a distortion of the upstroke or downstroke, combinations thereof, etc. The carotid pulse may be observed, recorded and compared with changes in various other metrics, such as changes with respiration and intra-thoracic pressure changes. The height of pulse pressure is roughly proportional to the ratio of the stroke volume to arterial distensibility. Arterial distensibility decreases as the distending pressure within the artery increases. Consequently, a given stroke volume will produce a larger pulse pressure if the mean arterial pressure is elevated. Arterial distensibility is also inversely related to the rate of rise of intraluminal pressure. As the rate of ventricular ejection accelerates, the arterial wall stiffens and the pulse pressure increases. The amplitude of the pulse pressure can also be modified by the “peripheral runoff.” An accelerated runoff will lower the diastolic pressure and result in higher amplitude of the pulse pressure.

While the upstroke of the carotid pulse reflects the driving force and vessel compliance, the downstroke reflects the distensibility of vessels and the peripheral resistance. After the aortic pressure curve peaks, it begins a decline as ventricular ejection slows and blood continues to flow to the periphery. During the initial phase of ventricular relaxation, there is a momentary reversal of blood flow from the compliant central arteries back toward the ventricle. With this reversal of flow, the aortic valves close. A notch on the descending limb of the aortic pressure curve is associated with this transient reversal of blood flow. The subsequent smaller secondary positive wave, or dicrotic peak (dicrotic from the Greek “double beat”), has been attributed to the elastic recoil of the aorta and aortic valve. Following this small wave, the aortic pressure declines as peripheral runoff continues.

Using a plurality of sensing devices (e.g., patch-module pairs as described above with respect to FIGS. 1 and 2A-2C, sensors 345 of wearable device 340 in FIG. 3), the techniques described herein provide a robust method for determining local pulse, hemorrhage and shock. In some embodiments, seven independent measurements are used that collectively provide a comprehensive view of subject physiology for determining early signs of oncoming issues such as hemorrhage, shock, dizziness, fatigue, heat injury, etc.

The seven independent measurements may include: (1) autonomic tone via heart rate variability (HRV), and others depending on where the devices are placed; (2) respiration, rhythm and effort, including various ways of measuring respiration such as via EMG; (3) photoplethysmogram (PPG) waveform from local SpO2 integration, where the waveform reflects changes in pulsatile arterial blood flow and should trend with changes in local pulse; (4) micro-movement analysis correlated with cardiac stroke volume and valve closures, which should also change with variation in central pulse; (5) electrodermal response and/or somatosensory response to indicate changes in autonomic activity and likely shock; (6) muscle tremor and core temperature changes, which should both change with shock and blood loss; and (7) ultrasound waveforms. The ultrasound waveforms may be enabled or captured using one or more down-facing micro-ultrasound transducers (e.g., in modules of patch-module pairs). Such down-facing micro-ultrasound transducers, when strategically placed near key vessels, provide another view of the arterial blood flow combined with changes in arterial compliance.

The above-described metrics, running independently and simultaneously, can increase the robustness of overall monitoring to extract early signs of problems emerging from a subject, and for extracting higher level functions from the subject. While various embodiments are described herein with respect to monitoring for hemorrhagic or hypovolemic shock, the techniques described herein may be used for various other types of feature extraction, including for detecting one or more of sneezing, coughing, vomiting, cramps, urination, defecation, postural changes, choking, salivation, tear formation, dry eye, dry mouth, sweating, itching, etc. By capturing key metrics, appropriate notifications may be generated and delivered to manage care of a subject (e.g., to initiate treatment procedures, adjust medications or therapies, etc.).

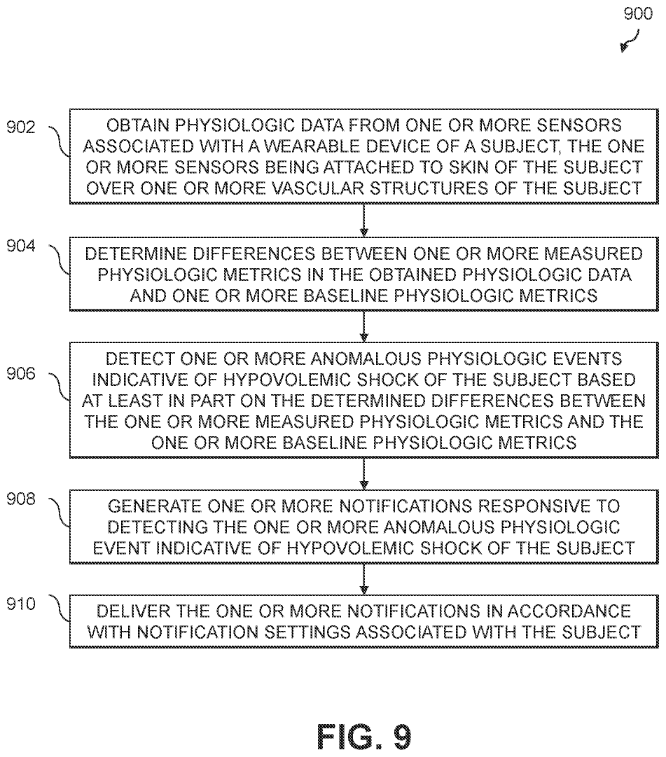

An exemplary process 900 for non-invasive detection of anomalous physiologic events indicative of hemorrhagic or hypovolemic shock will now be described with reference to the flow diagram of FIG. 9. It should be understood, however, that this particular process is only an example and that other types of processes for non-invasive detection of anomalous physiologic events indicative of hemorrhagic or hypovolemic shock may be used in other embodiments as described elsewhere herein. The process 900 includes steps 902 through 910, and is assumed to be performing by the health monitoring system 302 (e.g., utilizing the data processing module 320 and the notification module 322). In some embodiments, at least a portion of the process 900 may be performed by or using the wearable device 340.

The process 900 begins with step 902, obtaining physiologic data from one or more sensors (e.g., sensors 345) associated with a wearable device (e.g., 340) of a subject (e.g., 304-1), the one or more sensors being attached to skin of the subject over one or more vascular structures of the subject. In some embodiments, the one or more sensors are attached to the skin of the subject over one or more arteries and veins of a neck of the subject. The one or more sensors attached to the skin of the subject over the one or more vascular structures of the subject may be configured to independently measure: autonomic tone signals that are processed to determine HRV of the subject; local EMG signals that are processed to determine at least one of respiration rhythm and respiration effort of the subject; SpO2 signals that are processed to determine a PPG waveform characterizing changes in pulsatile arterial blood flow of the subject; movement signals that are processed to determine at least one of cardiac stroke volume and cardiac valve closures that change with variation in a pulse of the subject; at least one of electrodermal and somatosensory signals that are processed to indicate changes in autonomic activity of the subject; at least one of muscle tremor and core temperature signals that are processed to determine blood loss of the subject; and ultrasound waveform signals that are processed to determine arterial blood flow of the subject combined with changes in arterial compliance of the subject

In step 904, differences between one or more measured physiologic metrics in the obtained physiologic data and one or more baseline physiologic metrics are determined. One or more anomalous physiologic events indicative of hypovolemic shock of the subject are determined in step 906 based at least in part on the differences between the one or more measured physiologic metrics and the one or more baseline physiologic metrics determined in step 904. Step 906 may include detecting pulse volume of the subject correlated with at least one of a heart rate of the subject, a pulse rate of the subject, an amplitude of a pulse of the subject, a contour of the pulse of the subject, a pulse wave of the pulse of the subject, and a timing of carotid changes relative to heart sounds of the subject. Step 906 may also or alternatively include detecting at least one of a weak pulse and one or more trends in a pulse upstroke, downstroke and dwell of the subject from determined from a pulse contour of the subject.

In some embodiments, step 906 may further or alternatively include identifying one or more abnormalities in a carotid pulse of the subject, where the one or more abnormalities in the carotid pulse of the subject may comprise at least one of an alteration in an amplitude of a pulse peak of the carotid pulse, a distortion of a pulse upstroke of the carotid pulse, and a distortion of a pulse downstroke of the carotid pulse. Step 906 in such cases may further include correlating the identified one or more abnormalities in the carotid pulse of the subject with at least one of changes in respiration of the subject and changes in intra-thoracic pressure of the subject.

The process 900 continues with step 908, generating one or more notifications responsive to detecting the one or more anomalous physiologic event indicative of hypovolemic shock of the subject. In step 910, the one or more notifications are delivered in accordance with notification settings associated with the subject. In some embodiments, step 906 may include identifying that a difference between at least one of the one or more measured physiologic parameters and at least one of the one or more baseline physiologic metrics exceeds at least one designated threshold specified in the notification settings associated with the subject.

In some embodiments, step 908 may include selecting a notification type of a given one of the one or more notifications based at least in part on identifying that a difference between at least one of the one or more measured physiologic parameters and at least one of the one or more baseline physiologic metrics exceeds at least one designated threshold specified in the notification settings associated with the subject. Selecting the notification type may comprise selecting a type of stimulus to apply to the subject utilizing one or more indicator devices (e.g., indicator devices 346) associated with the wearable device. The selected type of stimulus may comprise at least one of an audible stimulus and a visual stimulus.

Step 910 may include delivering the one or more notifications to at least one of the wearable device and one or more additional devices. Delivering the one or more notifications to the wearable device may comprise selecting one or more indicator devices from a set of available indicator devices associated with the wearable device, and delivering the one or more notifications via the selected one or more indicator devices. The notification settings may comprise a first threshold for delivering the one or more notifications to the wearable device and a second threshold for delivering the one or more notifications to the one or more additional devices. The one or more additional devices may comprise one or more mobile computing devices associated with one or more users responsible for monitoring a health status of the subject.

In some embodiments, a wearable device for detecting early signs of hypovolemia comprises at least one sensor arranged to measure vascular signals from at least one carotid blood vessel, a controller in communication with the at least one sensor. The controller is configured to collect data from the at least one sensor, and to utilize a communication radio to transmit the data over a network to a monitoring system that analyzes the collected data to determine whether, where and how to deliver alerts to the wearable device (or to one or more other local or remote devices). The at least one sensor of the wearable device may comprise an electrocardiograph, ultrasonic transducer, plethysmograph or photoplethysmograph, sphygmomanometer, glucometer, thermometer, capnograph, means of measuring electrodermal activity such as somatosensory response or dermal impedance response, etc., configured to collect data about a subject. The monitoring system is configured to compare the collected data against baseline data to determine the likelihood an anomalous event will occur (or has or is occurring), and to trigger one or more notifications to be delivered to the wearable device, one or more local or remote devices, etc., to alert the operator or other user thereof to the anticipated onset of the anomalous event local device, or a remote device, alerting the operator of the device to the anticipated onset of the anomalous event (e.g., hemorrhagic or hypovolemic shock).

It will be appreciated that additional advantages and modifications will readily occur to those skilled in the art. Therefore, the disclosures presented herein and broader aspects thereof are not limited to the specific details and representative embodiments shown and described herein. Accordingly, many modifications, equivalents, and improvements may be included without departing from the spirit or scope of the general inventive concept as defined by the appended claims and their equivalents.

What is claimed is:

1. An apparatus comprising:
at least one processing device comprising a processor coupled to a memory;
the at least one processing device being configured:
to obtain physiologic data from one or more sensors associated with a wearable device of a subject, the one or more sensors being attached to skin of the subject over one or more vascular structures of the subject;
to determine differences between one or more measured physiologic metrics in the obtained physiologic data and one or more baseline physiologic metrics;
to detect one or more anomalous physiologic events indicative of hypovolemic shock of the subject based at least in part on the determined differences between the one or more measured physiologic metrics and the one or more baseline physiologic metrics;
to generate one or more notifications responsive to detecting the one or more anomalous physiologic event indicative of hypovolemic shock of the subject; and
to deliver the one or more notifications in accordance with notification settings associated with the subject.

2. The apparatus of claim 1, wherein the one or more sensors are attached to the skin of the subject over one or more arteries and veins of a neck of the subject.

3. The apparatus of claim 1, wherein detecting a given one of the one or more anomalous physiologic events indicative of hypovolemic shock of the subject comprises detecting pulse volume of the subject correlated with at least one of a heart rate of the subject, a pulse rate of the subject, an amplitude of a pulse of the subject, a contour of the pulse of the subject, a pulse wave of the pulse of the subject, and a timing of carotid changes relative to heart sounds of the subject.

4. The apparatus of claim 1, wherein detecting a given one of the one or more anomalous physiologic events indicative of hypovolemic shock of the subject comprises detecting at least one of a weak pulse and one or more trends in a pulse upstroke, downstroke and dwell of the subject from determined from a pulse contour of the subject.

5. The apparatus of claim 1, wherein detecting a given one of the one or more anomalous physiologic events indicative of hypovolemic shock of the subject comprises identifying one or more abnormalities in a carotid pulse of the subject.

6. The apparatus of claim 5, wherein the one or more abnormalities in the carotid pulse of the subject comprise at least one of an alteration in an amplitude of a pulse peak of the carotid pulse, a distortion of a pulse upstroke of the carotid pulse, and a distortion of a pulse downstroke of the carotid pulse.

7. The apparatus of claim 5, wherein detecting the given anomalous physiologic event indicative of hypovolemic shock of the subject comprises correlating the identified one or more abnormalities in the carotid pulse of the subject with at least one of changes in respiration of the subject and changes in intra-thoracic pressure of the subject.

8. The apparatus of claim 1, wherein the one or more sensors attached to the skin of the subject over the one or more vascular structures of the subject are configured to independently measure:
autonomic tone signals that are processed to determine heart rate variability of the subject;
local electromyographic signals that are processed to determine at least one of respiration rhythm and respiration effort of the subject;
oxygen saturation signals that are processed to determine a photoplethysmogram waveform characterizing changes in pulsatile arterial blood flow of the subject;
movement signals that are processed to determine at least one of cardiac stroke volume and cardiac valve closures that change with variation in a pulse of the subject;
at least one of electrodermal and somatosensory signals that are processed to indicate changes in autonomic activity of the subject;
at least one of muscle tremor and core temperature signals that are processed to determine blood loss of the subject; and
ultrasound waveform signals that are processed to determine arterial blood flow of the subject combined with changes in arterial compliance of the subject.

9. The apparatus of claim 1, wherein detecting a given one of the one or more anomalous physiologic events indicative of hypovolemic shock of the subject comprises identifying that a difference between at least one of the one or more measured physiologic parameters and at least one of the one or more baseline physiologic metrics exceeds at least one designated threshold specified in the notification settings associated with the subject.

10. The apparatus of claim 1, wherein generating a given one of the one or more notifications comprises selecting a notification type of the given notification based at least in part on identifying that a difference between at least one of the one or more measured physiologic parameters and at least one of the one or more baseline physiologic metrics exceeds at least one designated threshold specified in the notification settings associated with the subject.

11. The apparatus of claim 10 wherein selecting the notification type comprises selecting a type of stimulus to apply to the subject utilizing one or more indicator devices associated with the wearable device.

12. The apparatus of claim 10 wherein the selected type of stimulus comprises at least one of an audible stimulus and a visual stimulus.

13. The apparatus of claim 1, wherein delivering the one or more notifications in accordance with the notification settings associated with the subject comprises delivering the one or more notifications to at least one of the wearable device and one or more additional devices.

14. The apparatus of claim 13, wherein delivering the one or more notifications to the wearable device comprises selecting one or more indicator devices from a set of available indicator devices associated with the wearable device, and delivering the one or more notifications via the selected one or more indicator devices.

15. The apparatus of claim 13, wherein the notification settings comprise a first threshold for delivering the one or more notifications to the wearable device and a second threshold for delivering the one or more notifications to the one or more additional devices.

16. The apparatus of claim 13, wherein the one or more additional devices comprise one or more mobile computing devices associated with one or more users responsible for monitoring a health status of the subject.

17. The apparatus of claim 1, wherein the at least one processing device is part of the wearable device.

18. The apparatus of claim 1, wherein the at least one processing device is coupled to the wearable device over at least one network.

19. A computer program product comprising a non-transitory processor-readable storage medium having stored therein executable program code which, when executed, causes at least one processing device:
to obtain physiologic data from one or more sensors associated with a wearable device of a subject, the one or more sensors being attached to skin of the subject over one or more vascular structures of the subject;
to determine differences between one or more measured physiologic metrics in the obtained physiologic data and one or more baseline physiologic metrics;
to detect one or more anomalous physiologic events indicative of hypovolemic shock of the subject based at least in part on the determined differences between the one or more measured physiologic metrics and the one or more baseline physiologic metrics;
to generate one or more notifications responsive to detecting the one or more anomalous physiologic event indicative of hypovolemic shock of the subject; and
to deliver the one or more notifications in accordance with notification settings associated with the subject.

20. A method comprising:
obtaining physiologic data from one or more sensors associated with a wearable device of a subject, the one or more sensors being attached to skin of the subject over one or more vascular structures of the subject;
determining differences between one or more measured physiologic metrics in the obtained physiologic data and one or more baseline physiologic metrics;
detecting one or more anomalous physiologic events indicative of hypovolemic shock of the subject based at least in part on the determined differences between the one or more measured physiologic metrics and the one or more baseline physiologic metrics;
generating one or more notifications responsive to detecting the one or more anomalous physiologic event indicative of hypovolemic shock of the subject; and
delivering the one or more notifications in accordance with notification settings associated with the subject;
wherein the method is performed by at least one processing device comprising a processor coupled to a memory.
